(19) United States
(12) Patent Application Publication
(10) Pub. No.: US 2026/0204873 A1
(43) Pub. Date: Jul. 16, 2026

(54) TUNABLE VCSEL WITH ELECTRICAL AND OPTICAL CONFINEMENT VIA ETCHED POST

(71) Applicant: Bandwidth10, LTD., Berkeley, CA (US)

(72) Inventors: Carlos Fernando Rondina Mateus, Berkeley, CA (US); Christopher Chase, Berkeley, CA (US); Michael C. Y. Huang, Taipei (TW); Murat Serbay, Berlin (DE); Stefano Prandoni, Berlin (DE); Philip Worland, Berkeley, CA (US)

(21) Appl. No.: 19/428,495

(22) Filed: Dec. 22, 2025

Related U.S. Application Data
(63) Continuation-in-part of application No. 18/223,535, filed on Jul. 18, 2023, now Pat. No. 12,502,104.
Application No. 18/223,535 is a continuation-in-part of application No. 18/107,140, filed on Feb. 8, 2023, now Pat. No. 12,224,559.
Application No. 18/107,140 is a continuation-in-part of application No. 18/111,902, filed on Feb. 21, 2023.
Application No. 18/111,902 is a continuation-in-part of application No. 18/207,804, filed on Jun. 9, 2023, now Pat. No. 12,301,291.
Application No. 18/207,804 is a continuation-in-part of application No. 18/207,810, filed on Jun. 9, 2023, now Pat. No. 12,294,199.
Application No. 18/207,810 is a continuation-in-part of application No. 18/207,818, filed on Jun. 9, 2023, now Pat. No. 12,316,075.
Application No. 18/207,818 is a continuation-in-part of application No. 18/207,826, filed on Jun. 9, 2023, now Pat. No. 12,300,970.
Application No. 18/207,826 is a continuation-in-part of application No. 18/207,864, filed on Jun. 9, 2023, now abandoned.
Application No. 18/207,864 is a continuation-in-part of application No. 18/207,838, filed on Jun. 9, 2023, now Pat. No. 12,278,462.
Application No. 18/207,838 is a continuation-in-part of application No. 18/207,857, filed on Jun. 9, 2023, now Pat. No. 12,298,436.
Application No. 18/207,857 is a continuation-in-part of application No. 18/214,986, filed on Jun. 27, 2023, now Pat. No. 12,514,472.
Application No. 18/214,986 is a continuation-in-part of application No. 18/215,200, filed on Jun. 28, 2023, now abandoned.
(60) Provisional application No. 63/402,546, filed on Aug. 31, 2022.
(60) Provisional application No. 63/402,553, filed on Aug. 31, 2022.
(60) Provisional application No. 63/402,556, filed on Aug. 31, 2022.
(60) Provisional application No. 63/402,560, filed on Aug. 31, 2022.
(60) Provisional application No. 63/415,268, filed on Oct. 11, 2022.
(60) Provisional application No. 63/432,050, filed on Dec. 12, 2022.

(51) Int. Cl. H01S 5/183; H01S 5/10; H01S 5/343

(52) U.S. Cl. CPC H01S 5/18308; H01S 5/1042; H01S 5/18361; H01S 5/34306

(57) ABSTRACT

A light emitting apparatus includes a VCSEL laser. The VCSEL laser has one or more active regions with quantum wells and barriers. The active regions are surrounded by one or more p-n junctions. The one or more active regions can include a selected shape structure, as well as one or more tunnel junctions (TJ). One or more apertures are provided with the selected shape structure. One or more buried tunnel junctions (BTJ) or oxide confine apertured, additional TJ's, planar structures and or additional BTJ's are created during a regrowth process that is independent of a first growth process. A VCSEL output is determined in response to a monitoring application of the VCSEL. The VCSEL has an HCG grating and a bottom DBR. An etched post is between an active region and a sacrificial layer.

### CLAIMS OF PRIORITY AND CROSS REFERENCES TO RELATED APPLICATIONS

This patent application is continuation in part of U.S. Non-Provisional patent application Ser. No. 18/223,535, title ‘HEALTH-MONITORING DEVICE WIT VCSEL’, filed on Jul. 18, 2023, which claims priority from U.S. Provisional Application No. 63/402,546, title ‘HCG TUNABLE VCSEL WITH FULLY OXIDIZED BOTTOM DBR (GAAS BASED VCSEL)’, filed on Aug. 31, 2022; U.S. Provisional Application No. 63/402,553, title ‘HCG TUNABLE VCSEL WITH OPTICAL CONFINEMENT VIA STEP INDEX AND REGROWTH’, filed on Aug. 31, 2022, U.S. Provisional Application No. 63/402,556, title ‘HCG TUNABLE VCSEL WITH ELECTRICAL AND OPTICAL CONFINEMENT VIA ETCHED POST’, filed on Aug. 31, 2022; U.S. Provisional Application No. 63/402,560, title ‘FLIP CHIP TUNABLE VCSEL WITH HCG ON SI SUBMOUNT’, filed on Aug. 31, 2022; U.S. Provisional Application No. 63/415,268, title ‘LIGHT EMITTING DEVICE WITH DETERMINABLE SHAPE OF OUTPUT BEAM’, filed on Oct. 11, 2022 and U.S. Provisional Application No. 63/432,050, title ‘HCG TUNABLE VCSEL WITH INTEGRATED DETECTOR IN THE SACRIFICIAL LAYER’, filed on Dec. 12, 2022; is a continuation in part of U.S. Non-Provisional patent application Ser. No. 18/107,140, title ‘LIGHT EMITTING DEVICE WITH DETERMINABLE SHAPE OF OUTPUT BEAM’, filed on Feb. 8, 2023; U.S. Non-Provisional patent application Ser. No. 18/111,902, title ‘HCG TUNABLE VCSEL WITH ELECTRICAL AND OPTICAL CONFINEMENT VIA ETCHED POST’, filed on Feb. 21, 2023; U.S. Non-Provisional patent application Ser. No. 18/207,804, title ‘SYSTEMS FOR DETECTING GESTURES’, filed on Jun. 9, 2023; U.S. Non-Provisional patent application Ser. No. 18/207,810, title ‘SYSTEMS USING (3D) MAPS OF AT LEAST A PART OF A BODY OF A USER’, filed on Jun. 9, 2023; U.S. Non-Provisional patent application Ser. No. 18/207,818, title ‘SYSTEMS INCLUDING ONE OR MORE VCSELS, FOR RECEIVING A THREE DIMENSIONAL (3D) MAP OF AT LEAST A PART OF A BODY OF A USER’, filed on Jun. 9, 2023; U.S. Non-Provisional patent application Ser. No. 18/207,826, title ‘DIGITAL CAMERA WITH VCSELS’, filed on Jun. 9, 2023; U.S. Non-Provisional patent application Ser. No. 18/207,864, title ‘OPTOELECTRONIC DEVICE WITH VCSEL PROVIDING A GRID PATTERN’, filed on Jun. 9, 2023; U.S. Non-Provisional patent application Ser. No. 18/207,838, title ‘APPARATUS FOR FACIAL RECOGNITION’, filed on Jun. 9, 2023; U.S. Non-Provisional patent application Ser. No. 18/207,857, title ‘DEPTH MAPPING SENSING APPARATUS’, filed on Jun. 9, 2023; U.S. Non-Provisional patent application Ser. No. 18/214,986, title ‘HEALTH-MONITORING MEASUREMENT AND/OR MONITORING WITH A SMARTWATCH THAT INCLUDES ONE OR MORE VCSEL'S’, filed on Jun. 27, 2023; U.S. Non-Provisional patent application Ser. No. 18/215,200, titled ‘VCSEL WITH TUNABLE GRATING’, filed on Jun. 28, 2023.

### BACKGROUND

### Field the Invention

This invention related to HCG tunable VCSEL's, and more particularly to HCG tunable VCSEL with electrical and optical confinement via etched post.

### Brief Description of the Related Art

Vertical-cavity surface emitting lasers have been fabricated using selective oxidation to form a current aperture under a top monolithic distributed Bragg reflector mirror are reported. Large cross-sectional area lasers (259 μm2) exhibit threshold current densities of 150 A/cm2 per quantum well and record low threshold voltage of 1.33 V. Smaller lasers (36 μm2) possess threshold currents of 900 μA with maximum output powers greater than 1 mW. The record performance of these oxidized vertical-cavity lasers arises from the low mirror series resistance and very efficient current injection into the active region. Choquette, K. D. ; Schneider, R. P.; Lear, K. L. ; Geib, K. M.: ‘Low threshold voltage vertical-cavity lasers fabricated by selective oxidation’, Electronics Letters, 1994, 30, (24), p. 2043-2044, DOI: 10.1049/el: 19941421.

Vertical-cavity electrically driven lasers have been created with three GaInAs quantum wells and diameters of several μm exhibit room temperature pulsed current thresholds as low as 1·3 mA with 958 nm output wavelength. J. L. Jewell, A. Scherer, S. L. McCall, Y. H. Lee, S. J. Walker, J. P. Harbison, and L. T. Florez, “Low threshold electrically-pumped vertical-cavity surface-emitting micro-lasers,” Optics News 15(12), 10-11 (1989).

VCSEL's have been created with buried heterostructures. As a non-limiting example, a first buried active region vertical cavity surface-emitting laser diodes have been fabricated using in situ dry etching and molecular beam epitaxial regrowth. The laser emission of the etched/regrown devices persists over a greater current range and exhibit maximum output powers larger than air-post lasers. The lasers are anisotropically etched into the lower monolithic distributed Bragg reflector using an electron cyclotron resonance SiCl4 plasma etch. After transfer in ultra-high vacuum, epitaxial AlGaAs current blocking layers are regrown around the etched mesas. Polycrystalline deposition on the Si02 mask is removed by reactive ion etching to allow electrical contact and top surface emission. The etched/regrown laser characteristics demonstrate efficient current confinement and low thermal impedance. The vacuum integrated processing described here offers the prospect of further device performance enhancements and greater functionality. K. D. Choquette, M. Hong, R. S. Freund, J. P. Mannaerts, R. C. Wetzel and R. E. Leibenguth, “Vertical-cavity surface-emitting laser diodes fabricated by in situ dry etching and molecular beam epitaxial regrowth,” in IEEE Photonics Technology Letters, vol. 5, no. 3, pp. 284-287, March 1993, doi: 10.1109/68.205613.

To improve the performance of long-wavelength VCSEL's a study of tunnel junctions has been conducted for current injection and confinement. The introduction of a tunnel junction in long-wavelength VCSELs can substantially improve the current injection scheme, because an n-type material is utilized in the p-side region by inserting a tunnel junction. Long-wavelength lasers were fabricated with a tunnel junction formed by metalorganic chemical vapor deposition (MOCVD) using tertiarybutylphosphine (TBP) and tertiarybutylarsine (TBAs), and confirmed the current homogenization effect. Another proposed current confinement structure is by means of a simple fabrication process using a tunnel junction, i.e., the automatically formed tunneling aperture (AFTA). The AFTA has a tunnel junction aperture and can be formed automatically during electrode thermal annealing. Condition of the AFTA was investigated by varying the annealing temperature and time. Ortsiefer, Markus & Shau, Robert & Böhm, Gerhard & Köhler, Fabian & Abstreiter, Gerhard & Amann, Markus-Christian. (2000). Low-resistance InGa (Al)As Tunnel Junctions for Long Wavelength Vertical-cavity Surface-emitting Lasers. Japanese Journal of Applied Physics. 39. 1727-1729. 10.1143/JJAP.39.1727.

Optical lateral confinement in a VCSEL also plays an important role in improving its efficiency. The larger the overlap in between optical field and electrical current the more efficient the VCSEL will be. Current confinement along an aperture is a “must have” in VCSELs - lateral optical confinement is a more relaxed requirement (efficiency related). Some VCSEL designs for electrical current confinement, such as selective oxide aperture and buried tunnel junction (BTJ), also provide optical confinement due to the creation of a step index.

Oxide apertures are feasible only for short wavelength VCSELs, approximately up to 1060 nm, which are manufactured in the GaAs base system. This system allows Ga and Al atoms to be interchangeable in the lattice as AlAs, GaAs, and intermediary combinations of AlGaAs are lattice matched. High Al content in epitaxial layers (>90%) allows for selective oxidation (Al2O3) and creation of electrical apertures, as oxide is not electrically conductive. Such apertures preserve AlGaAs material in the center and the difference in index in between AlGaAs and Al2O3 also provides optical confinement (see Choquette 1994).

Long wavelength VCSELs (above 1.3 um, inclusive) do not have a material system which allows for reliable selective oxidation, mainly due to low quality oxides obtained from selective oxidation. These VCSELs have been using ion implantation as an alternative for current confinement in monolithically manufactured VCSELs. A similar effect can be achieved with buried tunnel junction (BTJ, see Ortsiefer 2000), if regrowth material around tunnel junction aperture has smaller index of refraction than the material in the tunnel junction (see Amann 2000).

Even though BTJ can be a better approach for current confinement, the process requires either long regrowth or wafer bonding techniques. Regrowth or bonding interfaces can bring reliability issues. Ion implanted VCSELs are monolithic and also provide the mesa good current confinement, but not optical confinement.

Some other design approaches which provide both electrical and optical confinement include buried heterostructures (see Choquette 1993) and etched air-post (see Jewel 1989).

The simplest way to transversely define the VCSEL cavity for both electrical and optical confinement is the etched air-post approach. Dry etch techniques, such as reactive ion etching or inductive coupled plasma etching can define small diameter air-posts with smooth side walls. A small diameter can define low active volume and thus small threshold currents and high modulation bandwidths.

An object of the present invention is to provide a VCSEL with an epitaxial structure that includes a post etch between an active region and a sacrificial layer.

Another object of the present invention is to provide a VCSEL with a regrowth of a sacrificial layer and HCG layer around an etched post.

Yet another object of the present invention is to provide a fully epitaxial grown tunable VCSEL with a very small cavity volume, lateral electrical current and optical confinement, and thus, increased efficiency.

These and other objects of the present invention are achieved in a light emitting apparatus with a VCSEL laser. The VCSEL laser has one or more active regions with quantum wells and barriers. The active regions are surrounded by one or more p-n junctions. The one or more active regions can include a selected shape structure, as well as one or more tunnel junctions (TJ). One or more apertures are provided with the selected shape structure. One or more buried tunnel junctions (BTJ) or oxide confine apertured, additional TJ's, planar structures and or additional BTJ's are created during a regrowth process that is independent of a first growth process. A VCSEL output is determined in response to a monitoring application of the VCSEL. The VCSEL has an HCG grating and a bottom DBR. An etched post is between an active region and a sacrificial layer.

### BRIEF DESCRIPTION OF THE DRAWINGS

FIG. 1A illustrates one embodiment of a light emitting device of the present invention with a seed layer and HCG layer.

FIG. 1B illustrates an embodiment of FIG. 1A with a sacrificial layer and deep proton implant.

FIG. 1C illustrates the FIG. 1B embodiment with the deep proton implant but with a buried tunnel junction.

FIG. 1D illustrates one embodiment of a light emitting device of the present invention with multiple-junction HCG tunability.

FIG. 2 illustrates an embodiment with several stepped BTJ apertures with different geometric shapes.

FIG. 3 illustrates a shaped structure, which can be a plateau, of the present invention.

FIG. 4 illustrates one embodiment of a P-N junction.

FIG. 5 illustrates an embodiment of FIG. 1A with an output beam and an optical beam inside of a cavity.

FIG. 6 illustrates one embodiment of electrical current confinement through a BTJ aperture, where electrical current flow from a top to a bottom contact.

FIG. 7 is similar to FIG. 3, but with a planarized HCG final layer.

FIG. 8 illustrates one embodiment of a manufacturing sequence with a growth of a bottom, a timed etch of the TJ, leaving some p++, and a regrowth on top of a VCSEL from an etched seed layer.

FIG. 9 illustrates one embodiment of a manufacturing sequence with a timed slow etch.

FIG. 10 illustrates one embodiment of a manufacturing sequence with a regrowth of n-InP with grading, and a regrowth of a top DBR, sacrificial layer and HCG layer.

FIGS. 11A and 11B illustrate a first regrowth including a bottom DBR, active layer (region), top DBR and other spacing layers.

FIGS. 12A and 12B illustrates one embodiment of implantation.

FIGS. 13A and 13B illustrate cross sections and top views of a mesa etch of the present invention.

FIGS. 14A and 14B illustrate cross sections and top views of ALD wall protection for further selective etch on release, reliability, of the present invention.

FIGS. 15A and 15B illustrate cross sections and top views of InP growth of the present invention.

FIGS. 16A and 16B illustrate cross sections and top views of the HCG layer of the present invention.

FIGS. 17A and 17B illustrate cross sections and top views of the HCG/MEMS litho/dry Etch of the present invention.

FIGS. 18A and 18B illustrate cross sections and top views of the MO-MEMs anchors, of the present invention.

FIGS. 19A and 19B illustrate cross sections and top views of the second mesa etch, M1 access of the present invention.

FIGS. 20A and 20B illustrate cross sections and top views of the second mesa etch, M1deposit, of the present invention.

FIGS. 21A and 21B illustrate cross sections and top views of the M1 cut of the present invention.

FIGS. 22A and 22B illustrate cross sections and top views of the M1 cut of the

FIGS. 23A and 23B illustrate one embodiment of a half-VCSEL cross section of the present invention.

FIGS. 24A and 24B illustrate one embodiment of VCSEL processing, of the present invention, with metal contacts (2), mesa etch, polyimide and bonding pads.

FIGS. 25A and 25B illustrate a cross-sectional view of a SI sub-mount.

FIG. 26 illustrates one embodiment of a flip chip of the present invention.

FIG. 27 illustrates one embodiment of a computing system used with artificial intelligence engine coupled to VCSEL system.

FIGS. 28A and 28B illustrate release of the present invention.

FIGS. 29A and 29B illustrate release, cut at MEMs beams, of the present invention.

### DETAILED DESCRIPTION

As used herein, the term engine refers to software, firmware, hardware, or other component that can be used to effectuate a purpose. The engine will typically include software instructions that are stored in non-volatile memory (also referred to as secondary memory) and a processor with instructions to execute the software. When the software instructions are executed, at least a subset of the software instructions can be loaded into memory (also referred to as primary memory) by a processor. The processor then executes the software instructions in memory. The processor may be a shared processor, a dedicated processor, or a combination of shared or dedicated processors. A typical program will include calls to hardware components (such as I/O devices), which typically requires the execution of drivers. The drivers may or may not be considered part of the engine, but the distinction is not critical.

As used herein, the term database is used broadly to include any known or convenient means for storing data, whether centralized or distributed, relational or otherwise.

As used herein a mobile device includes, but is not limited to, a cell phone, such as Apple's iPhone®, other portable electronic devices, such as Apple's iPod Touches®, Apple's iPads®, and mobile devices based on Google's Android® operating system, and any other portable electronic device that includes software, firmware, hardware, or a combination thereof that is capable of at least receiving a wireless signal, decoding if needed, and exchanging information with a server. Typical components of mobile device may include but are not limited to persistent memories like flash ROM, random access memory like SRAM, a camera, a battery, LCD driver, a display, a cellular antenna, a speaker, a BLUETOOTH® circuit, and WIFI circuitry, where the persistent memory may contain programs, applications, and/or an operating system for the mobile device. For purposes of this application, a mobile device is also defined to include a fob, and its equivalents.

As used herein, the term “computer” is a general-purpose device that can be programmed to carry out a finite set of arithmetic or logical operations. Since a sequence of operations can be readily changed, the communication device can solve more than one kind of problem. A communication device can include of at least one processing element, typically a central processing unit (CPU) and some form of memory. The processing element carries out arithmetic and logic operations, and a sequencing and control unit that can change the order of operations based on stored information. Peripheral devices allow information to be retrieved from an external source, and the result of operations saved and retrieved. Communication device also includes a graphic display medium.

As used herein, the term “internet” is a global system of interconnected computer networks that use the standard Network Systems protocol suite (TCP/IP) to serve billions of users worldwide. It is a network of networks that consists of millions of private, public, academic, business, and government networks, of local to global scope, that are linked by a broad array of electronic, wireless and optical networking technologies. The internet carries an extensive range of information resources and services, such as the inter-linked hypertext documents of the World Wide Web (WWW) and the infrastructure to support email. The communications infrastructure of the internet consists of its hardware components and a system of software layers that control various aspects of the architecture.

As used herein, the term “extranet” is a computer network that allows controlled access from the outside. An extranet can be an extension of an organization's intranet that is extended to users outside the organization in isolation from all other internet users. An extranet can be an intranet mapped onto the public internet or some other transmission system not accessible to the general public, but managed by more than one company's administrator(s). Examples of extranet-style networks include but are not limited to:

LANs or WANs belonging to multiple organizations and interconnected and accessed using remote dial-up.

LANs or WANs belonging to multiple organizations and interconnected and accessed using dedicated lines.

Virtual private network (VPN) that is comprised of LANs or WANs belonging to multiple organizations, and that extends usage to remote users using special “tunneling” software that creates a secure, usually encrypted network connection over public lines, sometimes vian ISP.

As used herein, the term “Intranet” is a network that is owned by a single organization that controls its security policies and network management. Examples of intranets include but are not limited to: s LAN, a Wide-area network (WAN) that is comprised of a LAN that extends usage to remote employees with dial-up access, a WAN that is comprised of interconnected LANs using dedicated communication lines, a Virtual private network (VPN) that is comprised of a LAN or WAN that extends usage to remote employees or networks using special “tunneling” software that creates a secure, usually encrypted connection over public lines, sometimes vian Internet Service Provider (ISP).

For purposes of the present invention, the Internet, extranets and intranets collectively are referred to as (“Network Systems”).

As used herein “Cloud Application” refers to cloud application services or “software as a service” (SaaS) which deliver software over the Network Systems eliminating the need to install and run the application on a device.

As used herein “Cloud Platform” refers to a cloud platform services or “platform as a service” (PaaS) which deliver a computing platform and/or solution stack as a service, and facilitates the deployment of applications without the cost and complexity of obtaining and managing the underlying hardware and software layers.

As used herein “Cloud System” refers to cloud infrastructure services or “infrastructure as a service” (IAAS) which deliver computer infrastructure as a service with raw block storage and networking.

As used herein “Server” refers to server layers that consist of computer hardware and/or software products specifically designed for the delivery of cloud services.

As used herein, the term “user monitoring”: (i) cardiac monitoring, which generally refers to continuous electrocardiography with assessment of the user's condition relative to their cardiac rhythm. A small monitor worn by an ambulatory user for this purpose is known as a Holter monitor. Cardiac monitoring can also involve cardiac output monitoring vian invasive Swan-Ganz catheter (ii) Hemodynamic monitoring, which monitors the blood pressure and blood flow within the circulatory system. Blood pressure can be measured either invasively through an inserted blood pressure transducer assembly, or noninvasively with an inflatable blood pressure cuff. (iii) Respiratory monitoring, such as: pulse oximetry which involves measurement of the saturated percentage of oxygen in the blood, referred to as SpO2, and measured by an infrared finger cuff, capnography, which involves CO2 measurements, referred to as EtCO2 or end-tidal carbon dioxide concentration. The respiratory rate monitored as such is called AWRR or airway respiratory rate). (iv) respiratory rate monitoring through a thoracic transducer belt, an ECG channel or via capnography, (v) Neurological monitoring, such as of intracranial pressure. Special user monitors can incorporate the monitoring of brain waves electroencephalography, gas anesthetic concentrations, bispectral index (BIS), and the like, (vi) blood glucose monitoring using glucose sensors. (vii) childbirth monitoring with sensors that monitor various aspects of childbirth. (viii) body temperature monitoring which in one embodiment is through an adhesive pad containing a thermoelectric transducer. (ix) stress monitoring that can utilize sensors to provide warnings when stress levels signs are rising before a human can notice it and provide alerts and suggestions. (x) epilepsy monitoring. (xi) toxicity monitoring, (xii) general lifestyle parameters, (xiii) sleep, including but not limited to: sleep patterns, type of sleep, sleep disorders, movement during sleep, waking up, falling asleep, problems with sleep, habits during, before and after sleep, time of sleep, length sleep in terms of the amount of time for each sleep, body activities during sleep, brain patterns during sleep and the like (xiv) body gesture, movement and motion (xv) body habits, (xvi) and the like.

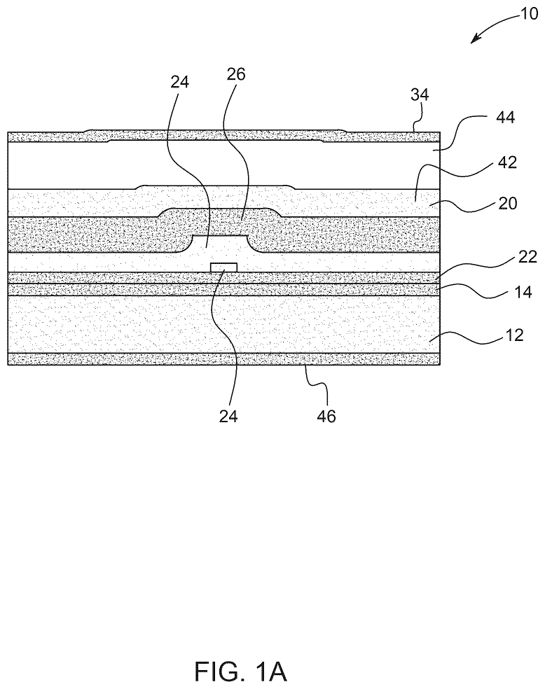

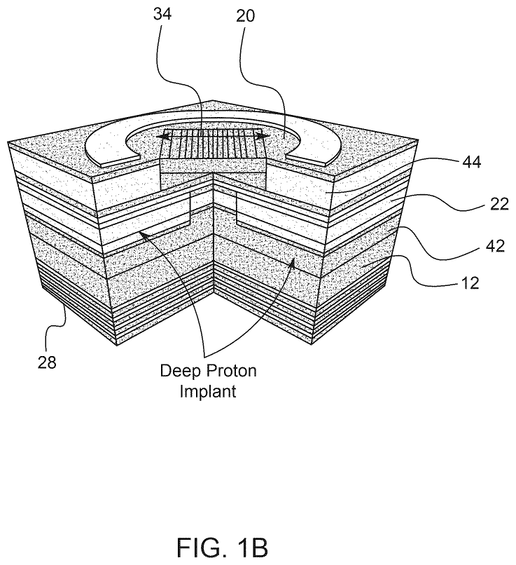

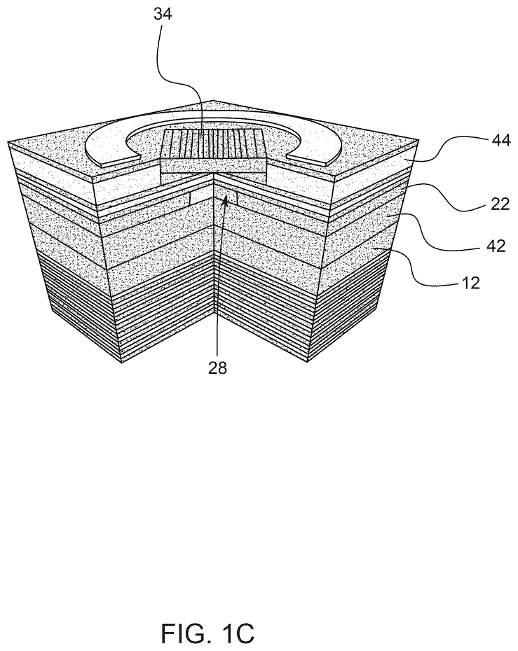

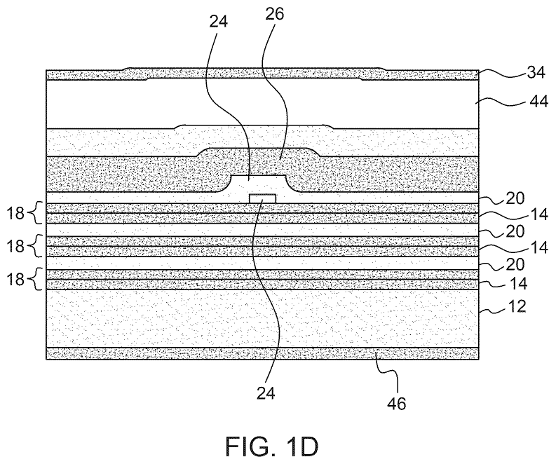

In one embodiment, illustrated in FIGS. 1A-1D, a light emitting device 10 includes:

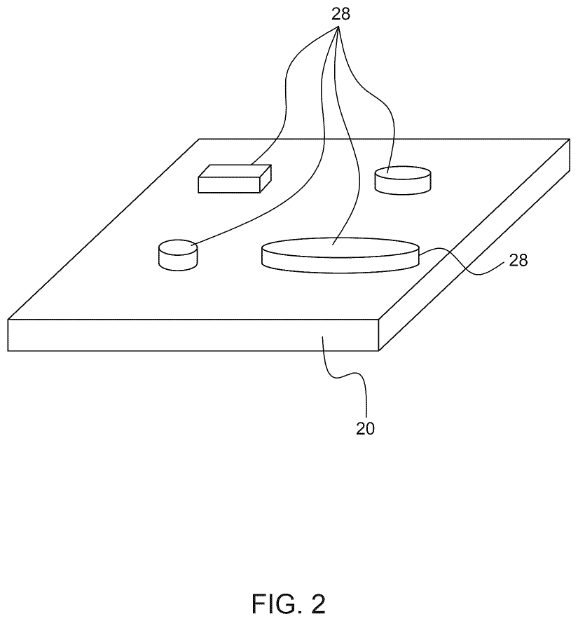

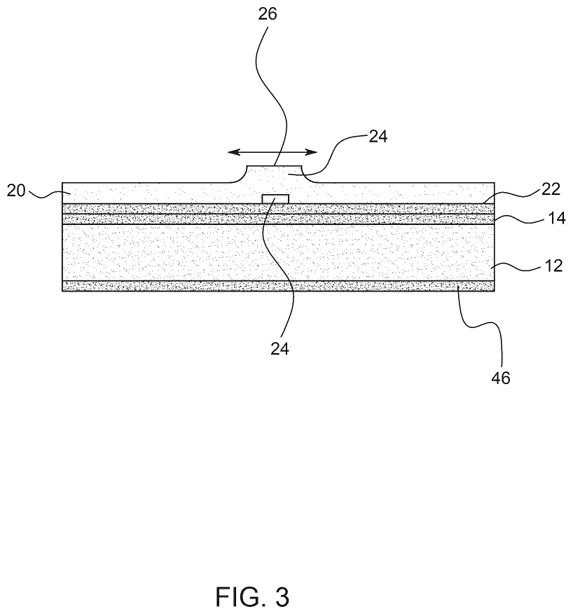

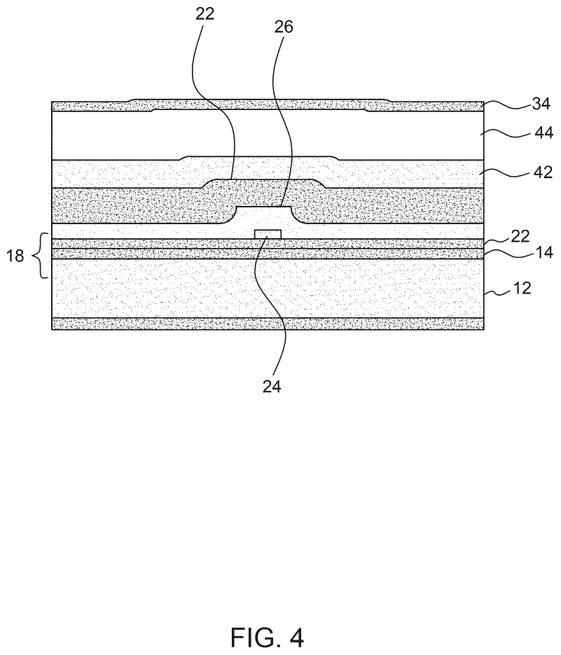

a first mirror 12 (“DBR”), which can be a bottom mirror, and one or more active regions 14. A first active region 14 is adjacent to the first mirror 12. The one or more active regions 14 are stacked in the light emitting device 10 to provide for a selected shape structure 26 with an active region that includes a tunnel junction (TJ) 20, another active region 14, another TJ 20 and so on. As illustrated in FIG. 2 there can be a repetition of active regions 14 and TJ's 20, providing a plurality of successive active regions 14 with TJ's 20, as illustrated in FIG. 2. Each active region 14 includes quantum wells and barriers 16 surrounded by one or more p-n junctions 18, see FIG. 3. As previously recited, one or more TJ's 20 are provided. Each of an aperture 24 is provided with a selected shaped structure 26, illustrated in FIG. 4. As non-limiting examples, the selected shape structure 26 can be open or closed, geometric or non-geometric shape, and the like. The shape of the selected shape structure 26 is based on a desired optical transmission function for a desired application, as shown in FIG. 4.

In one embodiment, the selected shape structure 26 is a stepped structure, see FIG. 4. The degree of the stepping can be at a variety of different angles, as determined by the etching process. The selected shape structure 26 can be substantially any geometry, including but not limited to a four corned star, circular, square and the like. As a non-limiting example, the selected shape structure 26 can be created using an inverse design. The selected shape structure 26 is selected in order to create an optimal desired far field pattern depending on the desired application. As a non-limiting example, the selected shape structure 26 is formed by etching.

In one embodiment, a first growth is used to create the bottom mirror 12, TJs 20, active regions 14, and the like. In one embodiment, a selected geometric configuration buried tunnel junction 28 is created during a regrowth process. The light emitting device 10 is created by the first growth, followed by a second or regrowth of the first growth. The first growth creates a planar structure. Additional BTJ's 28 are created from second, third and the like regrowth's. The creation of multiple BTJ's is formed during multiple regrowth processes. All of these regrowth's are independent of the first growth process.

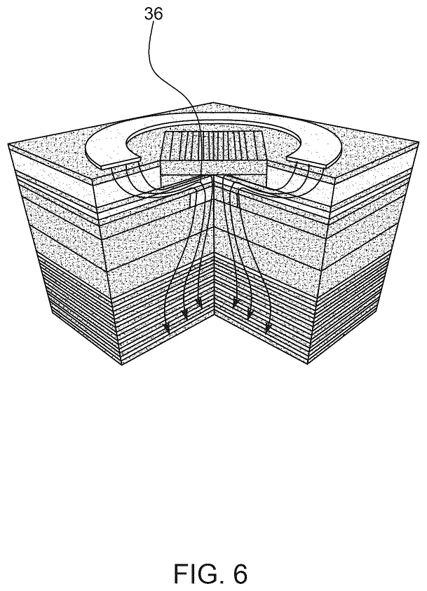

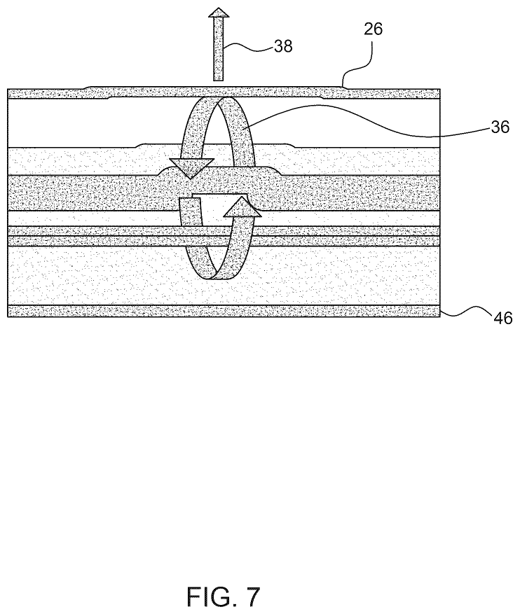

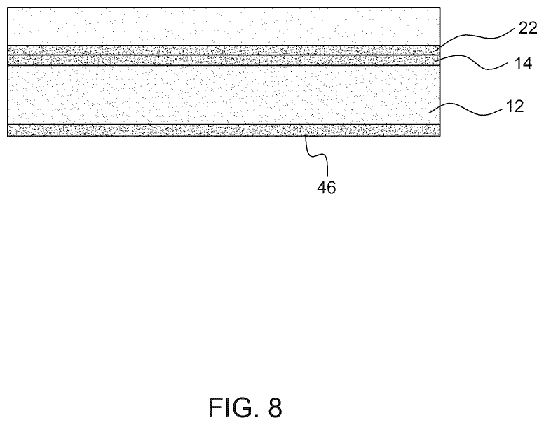

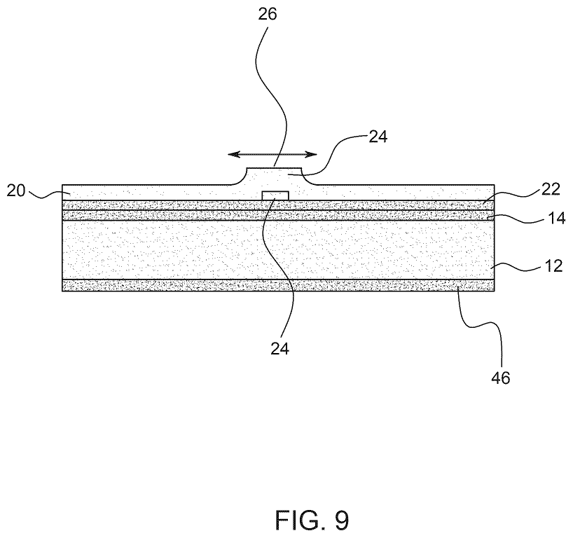

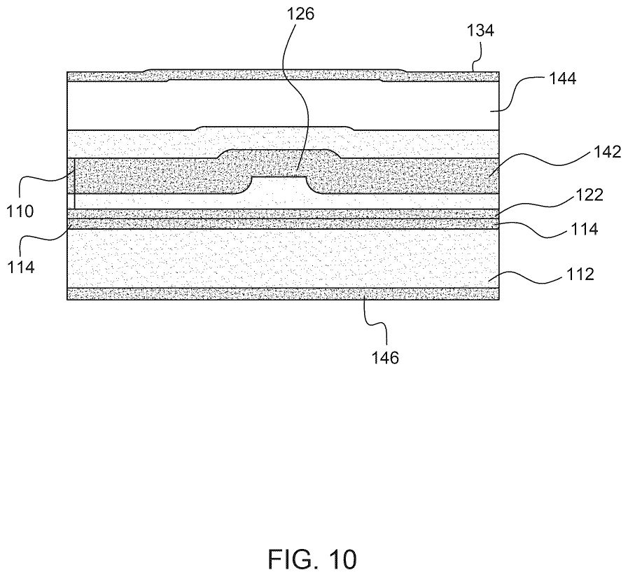

An area 30 is defined by the one or more BTJ's 28, additional TJ's 20, planar structures and/or addition BTJ's 28. A vertical resonator cavity 32 is defined by a second mirror over the electrical confinement aperture 30, see FIG. 6. A high contract grading (HCG) 34 operates as a second mirror positioned at the top of the vertical resonator cavity 32.

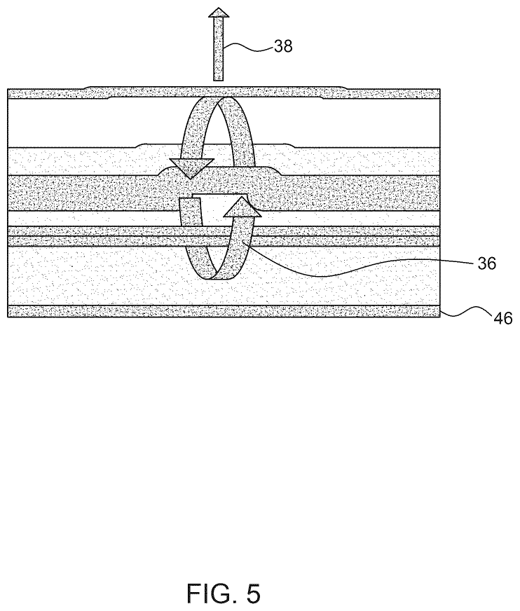

In one embodiment, the HCG 34 conformally covers the selected shape structure 26 layered on the selected shape structure 26. The HCG 34 remains anchored by a variety of different methods including not limited to: a membrane, or membrane structure, by beams after partial removal of sacrificial layer in order to move with respect to the optical resonator and modify a cavity length and consequent wavelength emitted by light emitting structure, and the like. A shape of the output beam 38, as in FIGS. 5 and 6, is determined by a geometric shape of the one or more BTJ apertures 29, and the apertures for additional TJ's 20, planar structures and/or additional BTJ's 28. and a transmission function of the HCG 34. HCG 34 is designed according to a desired optical transmission function for a desired application.

As a non-limiting example, the HCG 34 operates as a second mirror positioned over the vertical resonator cavity 32. In one embodiment, a shape of the output beam of light emitting device 10 is determined by a geometric shape of the one or more BTJ apertures 29, and apertures for additional TJ's 20, planar structures and/or additional BTJ's 28.

As a non-limiting example, the far field transmission range is application dependent. As a non-limiting example, the output beam can be a plurality of different geometries including but not limited to circular, elliptical and the like.

The selected shape structure can be created by a variety of ways. As non-limiting examples, the selected shape structure can be created can be made with a grid, a circular array of dots, a screen, hole and screens, and the like.

In one embodiment, the light emitting device 10 is a VCSEL (hereafter referred to as (“VCSEL 10”).

Relative to efficiency, the VCSEL 10 can be designed to maximize the light output relative to the desired application. Additionally, the number of BTJ's 28 created can be in response to the application.

In one embodiment, a shape of the output beam 38 is configured from a design of the electrical confinement aperture 30. As a non-limiting example, a geometry of the electrical confinement aperture 30 is slightly asymmetric to provide that the beam 36 inside the VCSEL 10 provides a Gaussian beam shape as the output beam 38 from the HCG 34.

In one embodiment, a coupling loss of the output beam 38 to a fiber or wavelength is reduced when VCSEL output beam 38 is designed to match the fiber modes. As a non-limiting example, the HCG 34 controls one or more of: an output beam 38 polarization, optical beam 3 shape and a single longitudinal wavelength. As a non-limiting example, the HCG 34 is further released from selected shape structure 26 by selective etch of spacing layers 22. In one embodiment, p-doping is on a top of the active region 14 leading to n-doping to a bottom of active region 14.

In one embodiment, at least a portion of the spacing layers 22 above the one or more BTJ apertures 29, the apertures for additional TJ's 20, planar structures and/or addition BTJ's 28, have thicknesses defined as multiples of quarter-wavelength of target lasing wavelength, in one embodiment, spacing layers and the top DBR 42 can have different thickness. As a non-limiting example, the top DBR 42 has a smaller number of layers than the bottom DBR.

Spacing layers 22 can also be used for electrical current spreading, thermal dissipation, or minimizing optical absorption, for example, but not limited to those ends.

In one embodiment, a top DBR 42 is not included in VCSEL 10. The sacrificial layer 44 of semiconductor material is removed via etching at specific locations in order to release the top mirror tuning structure. In one embodiment, the etching of the sacrificial layer 44 uses a wet or dry etch process. The sacrificial layer 44 can remain when the top mirror is not moveable as a non-tunable design and has a low index of refraction compared to a material of the top DBR 42. Successive steps are used in the growth of the VCSEL 10 and can also provide lateral optical confinement due to a graded lens effect. An epitaxial growth and a regrowth of the VCSEL 10 is achieved

In one embodiment, HCG 34 is formed of a semiconductor epitaxial layer. HCG 34 is layered on a selected shape structure 26, that can be less than 100 nm. In one embodiment, VCSEL 10 produces a stable linearly polarized output defined by HCG 34. In one embodiment of the present invention, VCSEL 10 is created by a process of two independent epitaxial growths. In one embodiment, first and second epitaxial growth structures are characterized, and formed by epitaxial growth.

As a non-limiting example, the first epitaxial growth structure is on a seed substrate 46 of III-V semiconductor material and includes: the bottom mirror Distributed Bragg Reflector (DBR) 26 on a top surface of the seed substrate 46 is defined by alternate layers of high and low index of refraction. Active region, generally denoted as 24, is adjacent to first mirror 12, consisting of quantum wells and barriers 16. A plurality of layers 22 can be optional. The final

layer, and the at least one TJ 20, are etched laterally to define an etched electrical confinement aperture 30 through which an electrical current flow. The HCG 34 and spacing layers 22 are formed over in a stepped format (selected shape structure 26). The etched electrical confinement aperture 30 being the one or more BTJs 28 that is a selected shape structure 26, as a result of further regrowth.

As a non-limiting example, the one or more BTJS 28 provide for VCSEL 10 current confinement in a VCSEL 10, and can be implemented for any VCSEL 10 in the SWIR band, from 650 to 1800 nm. More particularly, this can for InP based VCSELs 10 (above 1300 nm).

In one embodiment, for current confinement, the VCSEL 10 of the present invention uses the one or more BTJ's 28, additional TJ's 20, planar structures and/or addition BTJ's 2, instead of ion implant or oxide aperture.

As a non-limiting example, the VCSEL 10 with the one or more BTJ's 28, a BTJ 28 and can provide better current blocking outside of electrical confinement aperture 30 than ion implanted apertures and also provide good reliability.

In one embodiment, of the first epitaxial growth structure, the top surface of the seed substrate 46 is defined by alternate layers of high and low index of refraction.

In one embodiment, of the first epitaxial growth structure, the active region 14 is a source of light from an electrooptic effect due to a recombination of holes and electron. As a non-limiting example, the active region 14 is undoped and surrounded by one or more p-n junctions in order to promote recombination of electrons-holes.

In one embodiment, of the first epitaxial growth structure, at least a portion of the plurality of spacing layers 22 have a varied thicknesses depending on an optical design. In one embodiment, at least a portion of the plurality of spacing layers 22 are adjacent to the active region 14 or to additional spacing layers 22.

In one embodiment of the first epitaxial growth structure, the TJ 20 is a highly doped p++ layer directly on the top of a highly doped n++ layer.

In one embodiment of the first epitaxial growth structure, final layer is made of a same material of the seed substrate 46.

In one embodiment of the first epitaxial growth structure, the final layer is not included, depending on the optical design.

The TJ 20 layer is etched laterally and defines the selected shape structure 26, that is chosen based on the application. This becomes the one or more BTJs 28, a BTJ 28 additional TJ's 20, planar structures and/or addition BTJ's 28.

In one embodiment, a second epitaxial growth structure starts on a top of the first epitaxial growth structure after TJ 20 lateral etch. In one embodiment, the second epitaxial growth structure is a regrowth on top of the first stepped epitaxial growth structure, creating the one or more BTJs 28 from the one or more TJs 20, This becomes the one or more BTJs 28, additional TJ's 20, planar structures and/or addition BTJ's 28.

In one embodiment, the second epitaxial growth structure includes: spacing layers 22; an optional top DBR 42 mirror; a sacrificial layer; a top layer, which supports the top mirror manufacturing; and extra layers for supporting metal contacts.

In one embodiment, the p-doping is on a top of the active region 14 leading to n-doping to the bottom of the active region 14. In one embodiment, this is reversed and the n-doping is on the top of the active region 14, the p-doping is on the bottom.

In one embodiment of the second epitaxial growth structure, the second epitaxial growth structure starts on a top of the previously processed semiconductor stack.

In one embodiment of the second epitaxial growth structure, the second epitaxial growth structure includes spacing layers 22. As a non-limiting example, these spacing layers 22 have a varied thickness depending on the optical design.

In one embodiment of the second epitaxial growth structure, the spacing layers 22 are above the etched TJ 20 aperture. In one embodiment, the etch of the TJ 20 can be etching into the layers below the TJ 20. The TJ 20 and the layers below it can be fully etched, partially etched and the like. As a non-limiting example, the profile of the etch can be based of the selected application, and sidewalls can be created with sidewall angles of 20 to 90 degrees. The sidewalls of the etch can be created based on etching recipe, RF power, the type of chemicals used, crystal dependence, as well the desired end application. In one embodiment of the second epitaxial growth structure, they are added to complete a multiple of quarter-wavelength of a target lasing wavelength. In one embodiment, they have different thickness. In one embodiment, the spacing layers 22 are also used for electrical current spreading, thermal dissipation, or minimizing an optical absorption, and the like.

In one embodiment of the second epitaxial growth structure, the second epitaxial growth structure has a possible top DBR 42 mirror on top of a seed substrate 46, defined by alternating layers of high and low index of refraction of semiconductor. This top DBR 42 is not mandatory and, as a non-limiting example, has a much smaller number of layers when compared to bottom DBR 26, if it is included in the stack, and optimized based on the application, as well as two beam parameters. In one embodiment, the two beam parameters are reflectivity and the wavelength bandwidth of the mirror.

In one embodiment of the second epitaxial growth structure, the second epitaxial growth structure can be application specific, and include specific spacing sacrificial layer of semiconductor material under top mirror. This can be removed via etching at specific locations in order to release the top mirror tuning structure. In various embodiments, the etching of the sacrificial layer can be achieved with a wet or dry etch process. In one embodiment, this layer can alternatively remain in the structure if the top mirror is not supposed to move (non-tunable design) and has a low index of refraction compared to the top mirror material. In one embodiment, the index of refraction is application and can be as large as possible.

In one embodiment of the second epitaxial growth structure, the top mirror layer is processed during a post regrowth using an etching process and becomes a periodic structure designed to resonate and work as a mirror at specific wavelengths.

In one embodiment, the periodic structure is a high contrast metastructure (HCM) or high contrast grading HCG 34, known as subwavelength grading, or a photonic crystal structure. In one embodiment, the mirror effect requires a lower or a same index of refraction around the periodic structure, which can be air, if released, or other semiconductor or dielectric material, if not released. When it is a tunable structure, the periodic structure is actuated in order to change the optical length of optical cavity vian external input that causes movement of the periodic structure. In one embodiment, an external input is based on thermal, electrostatic or piezoelectric excitation. In one embodiment of the tunable design, tunability can also be achieved by heating the VCSEL 10 or changing its driving current.

In one embodiment of the second epitaxial growth structure, the second epitaxial growth structure extra layers are provided that are designed for supporting metal contacts.

In various embodiment, VCSEL 10 is at least one of: an air-cavity-dominant (ACD) or a semiconductor-cavity-dominant (SCD) design. In one embodiment, the regrowth process is configured to be nearly conformal and increase the lateral dimension of initial BTJ step as defined by the electrical confinement aperture 30 during deposition of epitaxial layers in the second epitaxial regrowth, in one embodiment, the regrowth process decreases a height of this step as more and more layers are added. In one embodiment, at the end, the periodic structure for the first mirror 12 is defined on a selected shape structure 26, which, however, does not have to be necessarily a plane.

As non-limiting examples, the successive steps can provide for lateral optical confinement due to a graded lens effect and improve overall efficiency of VCSEL 10.

In one embodiment, the epitaxial growth and regrowth of the VCSEL 10 can be achieved by conventional III-V epitaxy, such as molecular beam epitaxy (MBE) or metallo-organic chemical vapor deposition (MOCVD) or other techniques including but not limited to LPE, SPE and the like. While MBE can provide better control of each atomic layer, MOCVD can provide a better step coverage.

The HCG 34 can provide strong polarization control since the grading, HCG 34, acts as a polarizer, i.e., only stable linear polarization is transmitted through the grading, HCG 34, In one embodiment, the non-planar structure is formed due to the regrowth process which is started on selected shape structure 26 with TJ 20 delimited by a top of a selected geometric structure, including but not limited to a mesa.

In one embodiment, the one or more BTJ apertures 29 that are created during one or more regrowth's. After regrowth, etched TJ 29 becomes BTJ 20, a buried TJ.

In one embodiment, the HCG 34 is configured to reflect a first portion of light 36 back into the vertical resonator cavity 32. A second portion of light 38 is transmitted as an output beam from the light emitting structure 10. As a non-limiting example, every time the first portion of light 36 is reflected into the vertical resonator cavity 32, portions of light, constituting the second portion of light 38, come out of the light emitting structure 10. As a non-limiting example, the first portion of light 36 bounces around the vertical resonator cavity 36, with a portion coming out as the second portion of light 38.

As a non-limiting example, after sufficient thick regrowth, selected shape structure 26 can become planar, a plane plateau, and the like. It can easier to control processing of the periodic structure. In one embodiment, HCG 34 can be manufactured on a curved of stepwise layer, as far as continuous. In one embodiment, a final dimension of the plane plateau is originated from the epitaxial growth on top of electrical confinement aperture 30 and depends on a thickness of intermediary layers and growth conditions. As a non-limiting example, this thickness can be in the range of 10 um to 20 um for a starting electrical confinement aperture 30 with a 3-um diameter.

In one embodiment, VCSEL 10 is integrated in line detector behind it for FMCW applications, where the reflected light reflects into the VCSEL, causing optical feedback. The detector behind the VCSEL detects power oscillations due to the feedback and the signal can be used to reconstruct the 3D environment.

In one embodiment, a photodetector is at a side of an air gap in s tuning cavity as a detector. As a non-limiting example, the semiconductor, to the side of the air gap in the tunable structure, act as the photodetector and collect scattered light from the HCG and VCSEL body interface due to non-idealities. This offers some advantage to the application in 1) because the photodetector is outside of the cavity and does not contribute any additional feedback, simplifying the signal processing.

In one embodiment, a sensing apparatus includes a VCSEL 10 including one or more active regions 14. Each active region 14 has quantum wells and barriers 16. Active regions 14 are surrounded by one or more p-n junctions, the one or more active regions including a selected shape structure each with a tunnel junction (TJ). One or more apertures are provided with the selected shape structure. One or more buried tunnel junctions (BTJ) 28 or oxide confine aperture, additional TJ's 20, planar structures and/or additional BTJ's 28 are created during a regrowth process that is independent of a first growth process. An output of VCSEL 19 is determined in response to an application of the light emitting device. In one embodiment, bottom DBR 12 can include an HCG and dielectric coating with one or more layers of the dielectric coating.

In one embodiment, the dielectric coating is a stack of high/low index layers As non-limiting examples, the dielectric coating can be a stack of 5-10, 6-10, 7-10, 10 high/low index pairs of dielectric. The dielectric provides a wider tuning range. This provides a broad wavelength mirror. In one embodiment, the top DBR mirror 42 is s a moveable mirror.

With tunable, a specific wavelength is targeted and VCSEL 10 remains at that wavelength. Changing cavity 32 size allows VCSEL 10 to move, and physically change the cavity 32 size for each application In one embodiment, wavelength locking control is provided. This requires an external reference including but not limited to a chip that taps the light. This is then coupled to a reference system. The reference system and the chip are external to the VCSEL In one embodiment, atomic plots are used for the reference system.

As non-limiting examples, ranges of output power can be: one additional active region (or junction) can add ~1× power, and the laser could become 2× more powerful. However, as it also adds extra heat coming from additional optical power inside cavity, efficiency goes down. Thus, by adding more junctions we can have more power but to the price that the laser cannot work continuously anymore. Typical multi-junction VCSELs work on pulsed mode and it's very hard to predict range of output power. As a rule of thumb, we can say that 2 junctions will have 2× more power on pulsed operation, 3 junctions 3×. As one increases the number of junctions, pulses have to become faster and intervals longer.

In one embodiment, the output power is from 1 milliwatt to 5 milliwatts per aperture. In one embodiment, multi junction devices have output powers of 10-20 milliwatts, 12-17 milliwatts, and 15 milliwatts. In one embodiment, the oxide confinement defines aperture 24 for electrical current confinement.

As a non-limiting example, a number of quantum wells can be the same in all junctions, but not necessarily. Individual quantum well design can also be slightly different in order to broaden spectral material gain and increase tuning range.

### Method of VCSEL Formation

In one embodiment, a regular VCSEL apparatus 10 includes current injection via the one or more BTJs 28, TJ's 20, planar structures and/or additional BTJ's without ion implantation.

As a non-limiting example, VCSEL apparatus 10 includes one or more BTJ's 28 and is tunable. In one embodiment, formation of the one or more BTJ's 28 TJ's 20, planar structures and/or additional BTJ's, requires an etch of a TJ aperture and regrowth of a top semiconductor epitaxial structure. As a non-limiting example, the process results on a non-flat surface, not suitable for an HCG 34. Moreover, uneven surface and regrowth process causes additional strain which also prevents MEMS manufacturing. In one embodiment, this requires relaxation of epitaxial growth through careful design of regrowth and MEMS actuation on the non-planar surface.

In one embodiment, VCSEL 10 formation includes the steps of:
(i) growing a semiconductor active cavity material consisting of a multi-quantum well layer stack sandwiched between bottom and top spacer regions, the top spacer region terminating with a p-layer and a p++/n++ tunnel junction grown on top of the p-layer, each of the p++—and p-layer presenting a p-type layer;
(ii) etching the active cavity material formed in step (i) to form a top of a selected geometric structure such as a mesa, including at least the upper n++ layer of the tunnel junction emerging from the underlying p-type layer, thereby creating a structured surface of the active cavity material formed by the upper surface of the upper surface of the p-type layer outside a top of a selected structure such as a mesa;
(iii) applying a wafer fusion between the structured surface of the active cavity material and a substantially planar surface of a n-type semiconductor layer of a first distributed Bragg reflector (DBR) stack, thereby causing deformation of the fused surface around a top of a selected structure such as a mesa, and defining an electrical confinement aperture 30 for electrical current flow therethrough, the electrical confinement aperture 30 including a top of a selected structure such as a mesa, surrounded by an air gap between the deformed fused surfaces and defining an active region 14 of the device;
(iv) forming a second DBR stack on the surface of the active cavity material opposite to the structured surface;
(v) forming ohmic contacts on the VCSEL device structure to enable the electrical current flow through the electrical confinement aperture 30 to the active region 14.

In one embodiment, VCSEL apparatus 10 is made with the following processes: epitaxial growth of VCSEL up to tunnel junction (TJ) layer 20; development of TJ etches; identify etchants compatible to the TJ design 20; and development of regrowth process.

Surface compatibility to regrowth—not all surfaces or all materials are suitable for seeding the regrowth process—there is a trade off in between the best design for the TJ 20 and a design good enough which is suitable for regrowth.

Regrowth has to be carried long enough in order to planarize the surface where HCG 34 is manufactured on one or more of: VCSEL regular processing; growth of bottom VCSEL and then: etch of TJ 20, leaving some p++—target is 5-10 nm and regrowth of top VCSEL from etched seed layer.

Then there is: a regrowth of n-InP with grading; a regrowth of top DBR 42 (if included in design), sacrificial layer and HCG 34; followed by standard VCSEL manufacturing.

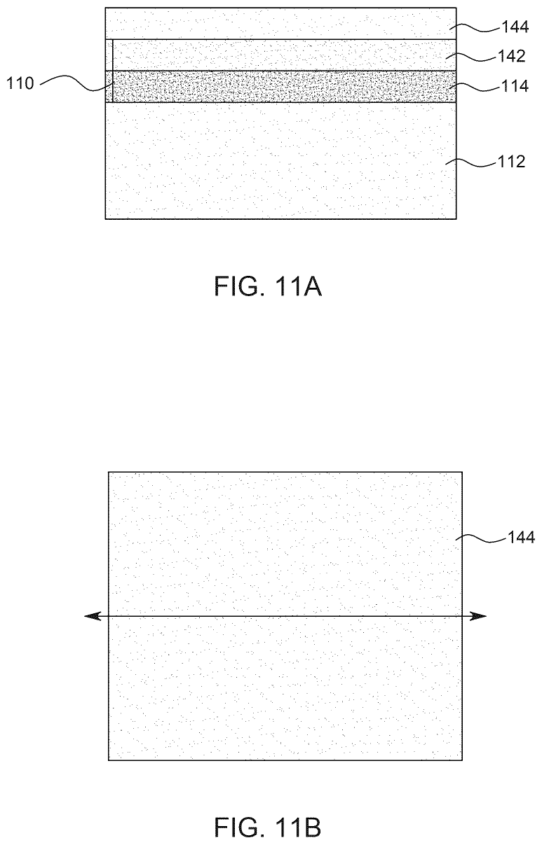

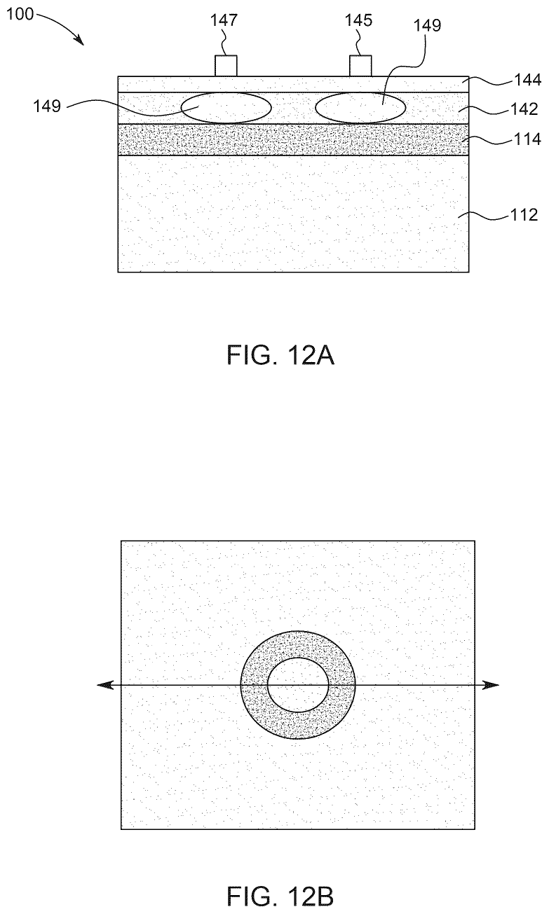

In one embodiment a regular VCSEL 10 epitaxial structure includes an etched post between an active region 112 and a sacrificial layer 114. FIGS. 11A and 11B illustrate a first regrowth including a bottom DBR 116, active layer (region) 112, top DBR 118/other spacing layers 120. The sacrificial layer 114 has a thickness now greater than 100 nm. The optical confinement is defined by the etched post 110 which acts as a waveguide. FIGS. 12A and 12B illustrates one embodiment of implantation.

In one embodiment, an etched post 110 and regrowth provide lateral current and optical confinement, small volume and increased efficiency for more demanding applications, such as very high-speed modulation and coherent communication. The increased efficiency is achieved because the optical wave and the lateral currents overlap.

Instead of etching the post 110 for confinement in the optical path via mesa etch and regrowth, the optical path is preserved and modify its boundaries for optical confinement. The existence of the sacrificial layer 114 in the present invention favors this new approach as the final interface of regrown material in the optical path is etched away. This preserves optical quality. As a non-limiting example, the manufacturing requires integration and compatibility of several different processes, not required for the conventional semiconductor as-grown DBR.

Regrowth of the sacrificial layer 114 around a small mesa 122 step brings much smaller complication when compared to previous approaches of buried heterostructures with steep walls more than 2× taller (3-4 um) than mesa 122 (0.8 um). In one embodiment, a full VCSEL is grown up to a thin SAC layer (100 nm of Al0.22Ga0.25In0.53As with 100 nm InP cap). As illustrated in FIGS. 12A and 12B implantation is then proceeded for current confinement under the metal contact pads. In illustrates the etched post 110.

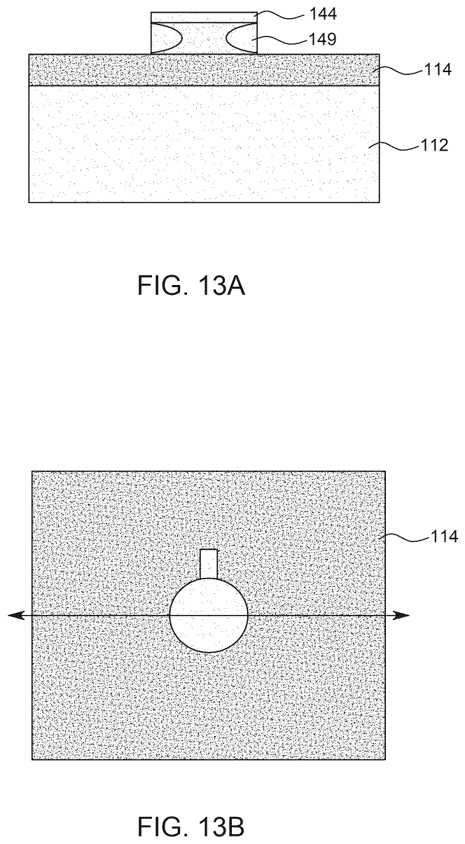

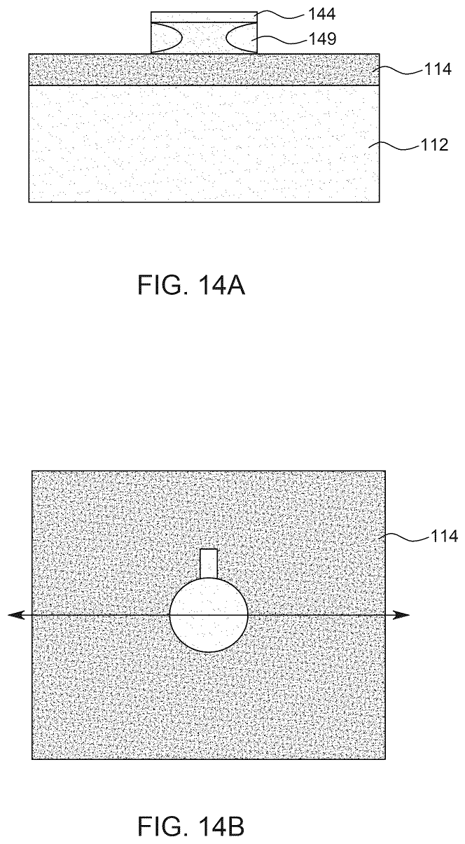

In one embodiment, implantation is done on a structure with the bottom DBR 116, active layer 112 and a thin sacrificial layer 114. The implantation is done on a layer between the active layer 112, and the sacrificial layer 114. An implant mask is used. The structure, including the bottom DBR 116, active layer 112, sacrificial layer 114 and mesa 122 position therebetween, is then dry etch down a very tiny mesa 122 for high-speed (radius of 5-10 um), up to InP layers, close to a TJ interface FIGS. 13A and 13B. The mesa 122 having an increasing radius up to a top layer. Optical monitoring can be used. Referring to FIGS. 14A and 14B, atomic layer deposition (ALD) is performed only on side walls of etched post 110. This protects the etched post 110 from further release, increased reliability and isolation from surrounding air.

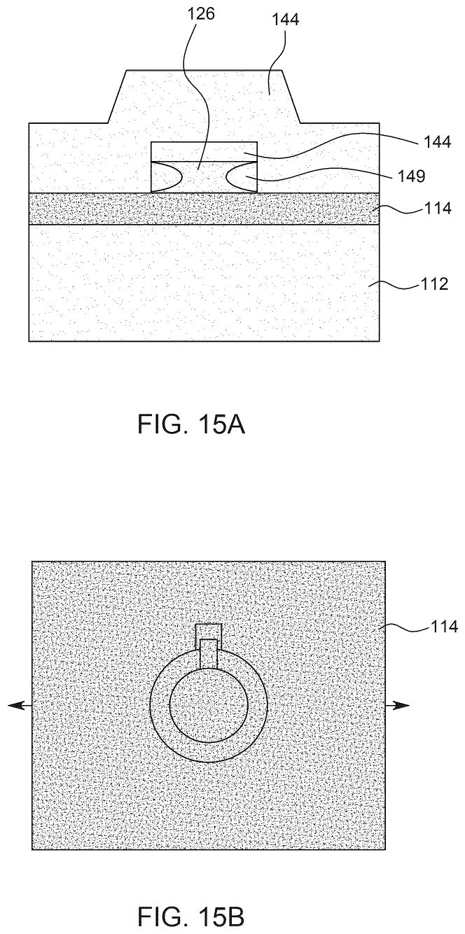

In one embodiment, illustrated in FIGS. 15A and 15B, ALD wall protection is used for further selective etch on release, and for reliability.

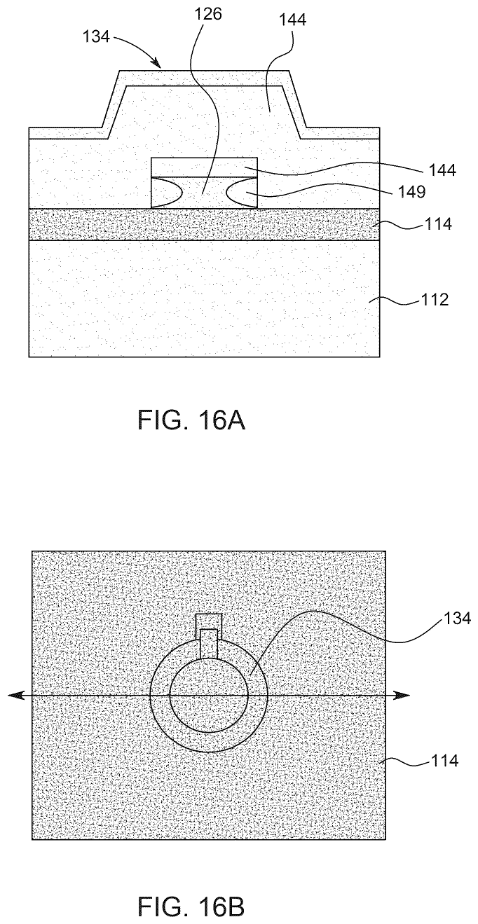

A full regrowth of a full sac layer+HCG on top is performed, as illustrated in FIGS. 16A and 16B, with the interface etched away. InP regrowth is a regrowth around the etched post 110. This is later removed, along with the sacrificial layer 114.

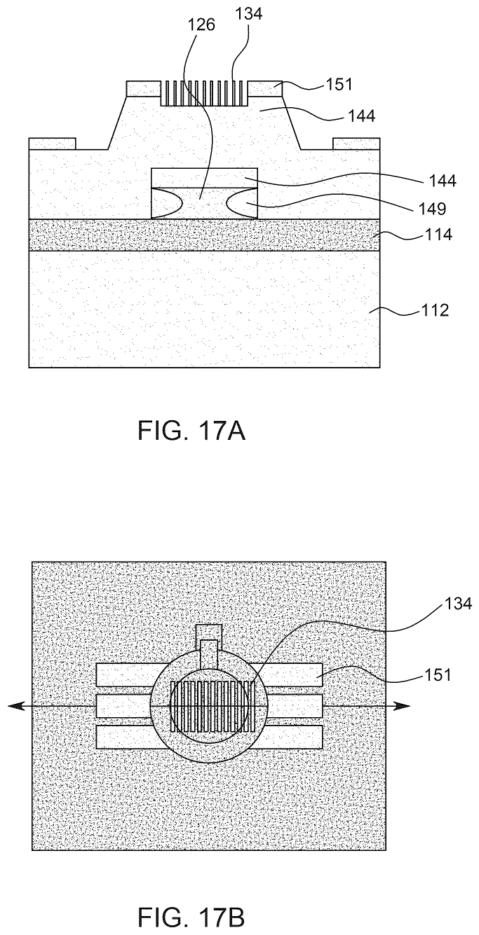

The regrowth interface is out of the optical/current paths. In one embodiment, the regrown layer, the new growing layers cause some defects on the crystal before arriving at the HCG layer. The structure includes the bottom DBR 116, active region (layer) 112, a thin sacrificial layer 114, a mesa 122 therebetween, and a regrowth sacrificial layer 114. In one embodiment, the mesa 122 grows laterally during regrowth for a slightly bigger than mesa 122 HCG, FIGS. 17A and 17B As a non-limiting example, a vertical step on the MEMS beams is not a problem. There is no need for implantation confinement.

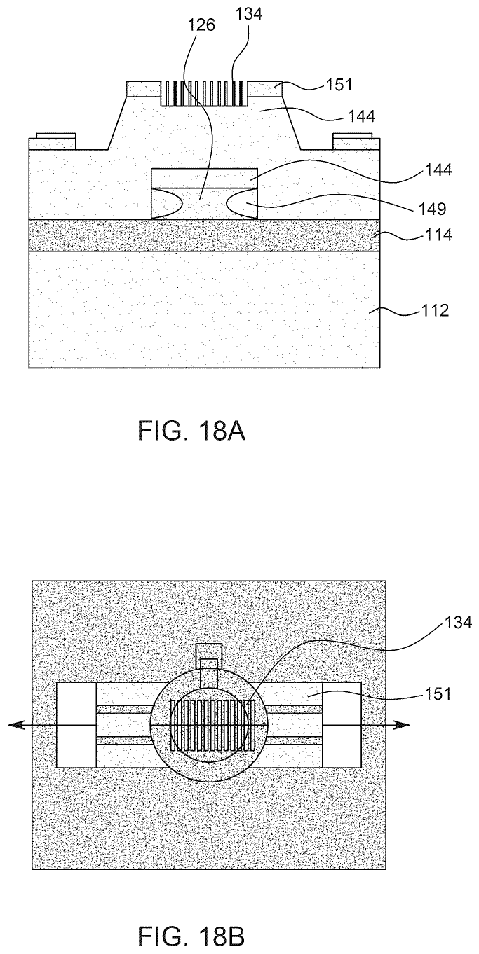

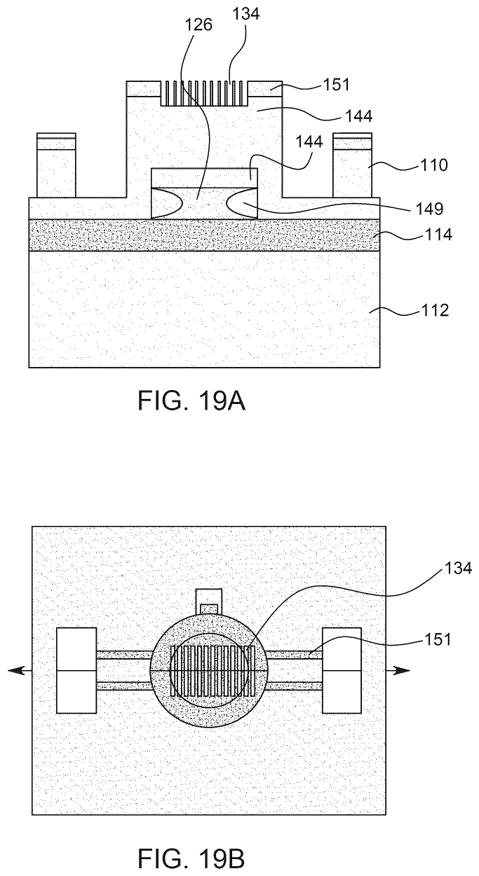

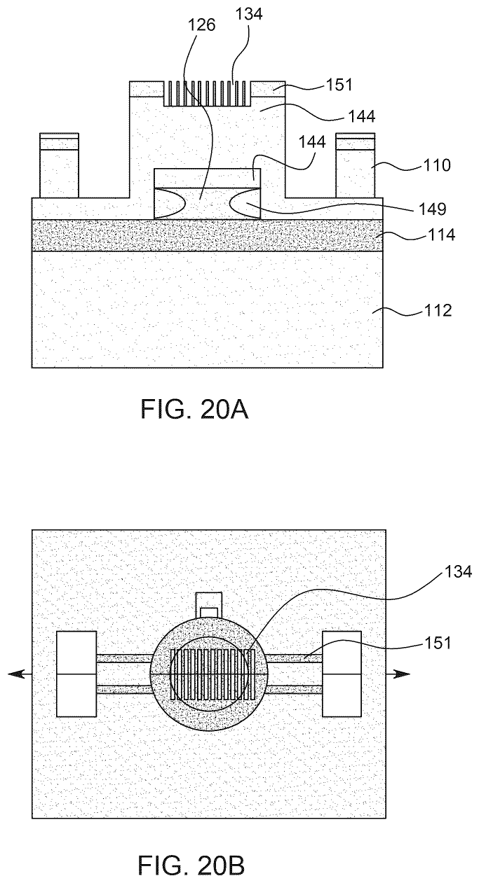

In one embodiment, mesa 122 confinement is provided, e.g, implantation is required only for contact isolation and current injection from the top of the mesa 122. In one embodiment, normal processing is completed for adding contact pads, which as a non-limiting example, the contact pads can be metal 3 and polyimide. MEMS anchors are on either side of the mesa 122, FIGS. 18A and 18B. In one embodiment, a second mesa etch is provides, FIGS. 19A and 19B, following by an MI cut, FIGS. 20A and 20B.

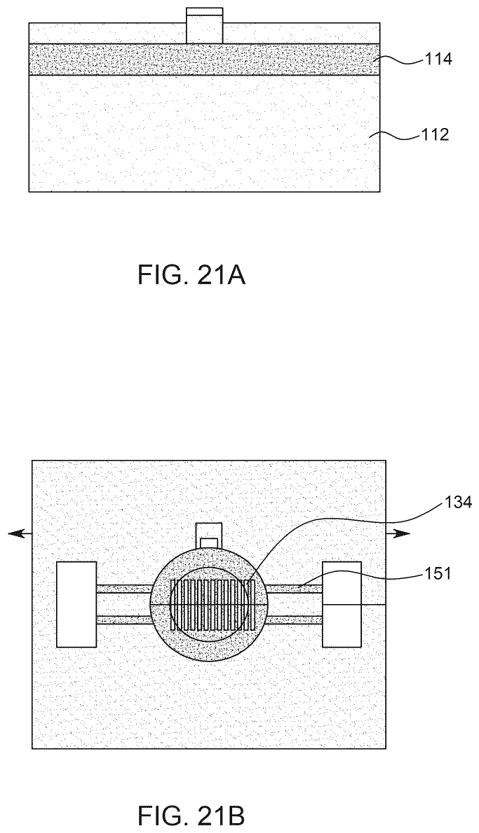

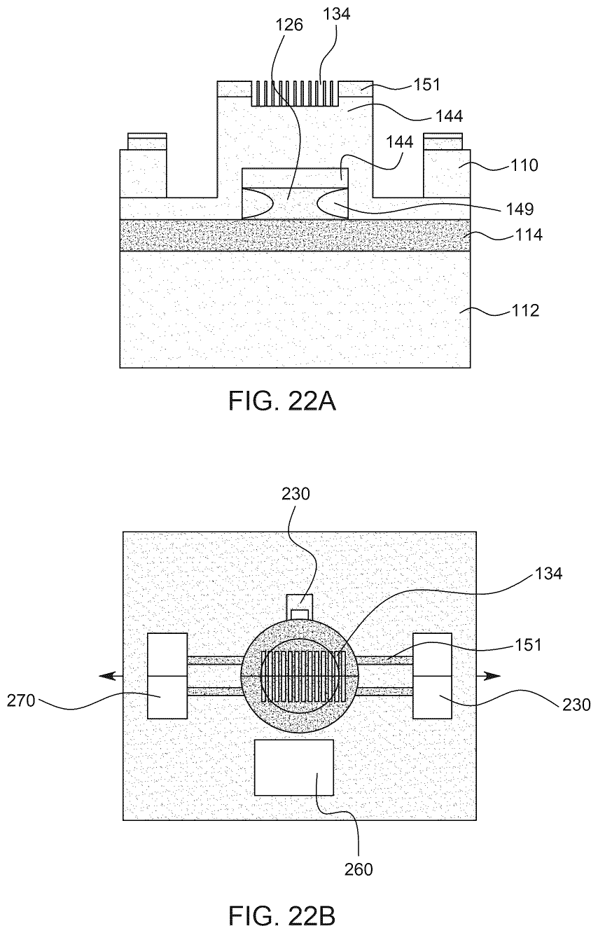

An opened access, M2, to the bottom DBR 116 contact is created, FIGS. 21A and 21B. This is followed by a release, FIGS. 22A and 22B. The release can be at the MEMS beams.

In one embodiment, the 38 of the VCSEL laser has a long wavelength, including but not limited to from 0.720 micron to 1.7 microns, and in one instance, 1.365 microns.

In one embodiment, VCSEL 10 products an output beam with a wavelength of 1550 nm is chosen due to its high signal-to-noise ratio (SNR) for glucose signals.

In one embodiment, the output 38 of the VCSEL laser is a long wavelength, at least partially created from indium phosphide structure or material in the VCSEL laser 10. As a non-limiting example, VCSEL laser 10 can include an indium phosphide substrate 46, or a substrate 46 that at least partially includes indium phosphide.

In one embodiment, VCSEL laser 10 is coupled to or includes a top DBR 42 or high contrast grading (HCG) 34. As a non-limiting example, bottom DBR 12 is a semiconductor DBR or a combination of a semiconductor DBR with a dielectric coating. In this manner, VCSEL 10 can include a dielectric coating. As a non-limiting example, the dielectric coating improves a broadening of a tuning range of the VCSEL laser 10.

In one embodiment, VCSEL laser 10 operates in a single mode or a multi-mode operation. As a non-limiting example, dimensions of the aperture and HCG 34 are contributing factors to the single mode operation.

In one embodiment, VCSEL laser 10 can deploy multiple tunnel junctions to enhance the output 38 of VCSEL laser 10.

As a non-limiting example, buried tunnel junctions (BTJ) improve an energy efficiency of VCSEL laser 10.

As a non-limiting example, a wavelength of the VCSEL laser output 38 can be swept to provide improved resolution. In one embodiment, the VCSEL laser output 38 is swept by modulating an HCG grading 34 up and down, e.g, its up and down movement relative to a top of VCSEL laser 10, wherein when the HCG 34 moves closes to non-extended portion relative to a top of VCSEL laser 10, a wavelength of the output 38 changes and returns closer to an original output 38 of the VCSEL laser, without extension of the grading.

In one embodiment, a mem's structure is coupled to the HCG grading 34 or top DBR 42 to create a swept source. As a non-limiting example, modulating a VCSEL laser output 38, in combination with a sweeping of wavelengths of the VCSEL laser output 38 allows for higher resolution and reduces at least a portion of atmospheric interference of VCSEL laser operation.

In one embodiment, multiple tunnel junctions increase an optical power of the VCSEL laser 19. These junctions are provided in a body of the VCSEL laser 10.

In one embodiment, a semiconductor optical amplifier is included with a modulator to allow a swept source to be modulated. As a non-limiting example, the modulation of the VCSEL laser turning the VCSEL laser on and off. As a non-limiting example, the modulation is from about 1 to 50 G. In another embodiment, the modulation is greater than 50 G.

As a non-limiting example, an optical photonic integrated circuit (PIC) is coupled to the VCSEL laser. The VCSEL laser can be mounted on the PIC. In one embodiment, a plurality of VCSEL lasers are mounted on the PIC.

In various embodiments, VCSEL 10 can have HCG 34 top and bottom structures with the high contract grading (HCG) 34. In one embodiment, the HCG 34 can be on a Si substrate or other such structures with the HCG 34 in the substrate and the VCSEL bond to the surface. In one embodiment, the HCG 34 can contain two or more PNG or NPN junctions, allowing the HCG to be bi directional. In one embodiment, the HCG 34 can have selected dimensions for pressure measurement, with a cochlea implant, with catheters and the like. VCSEL 10 can one or more junctions, with various wavelengths including but not limited to UV to IR.

In one embodiment, HCG 34 acts as a partial mirror. As a non-limiting example, HCG 34 can operate as a second mirror HCG 34 is positioned at the top of the vertical resonator cavity 32, following removal of at least a portion of the sacrificial layer 44. HCG 34 is positioned at the top of the vertical resonator cavity 32. As a non-limiting example, HCG 34 is positioned on a top of the mesa, in one embodiment, a plurality of support elements, anchors, hold HCG 34 in place. HCG 34 then moves, typically up and down relative to cavity 32.

Two or more support elements are provided. Support elements provide support for HCG 34. As a non-limiting example, support elements can have dimensions of 50 microns long, one micron wide. Their thickness is determined by a growth of HCG 34 layer. Sacrificial layer 44 is defined by epitaxial growth. As a non-limiting example, HCG deformation is provided under different points of operation. For broad tuning, HCG 34 deforms and moves away from cavity 32. A period and duty cycle of HCG 34 are determined by the amount of deformation. As a non-limiting example, a thickness of support elements define their function. A modification of support elements occurs when there is an actuation. This causes support elements to bend and HCG 34 deforms. In one embodiment, the support elements 45 are not straight beams. In various embodiments, there can be variations of support elements to modify HCG 34 deformation.

A good portion of sacrificial layer 44 is substantially removed, at least a portion creating anchors for HCG 34.

As a non-limiting example, a main actuation of HCG 34 can include but not be limited to: electrooptic, with an application of voltage across the surface of HCG 34, thermal, piezoelectric and the like, an air gap is between the top surface of vcsel laser 10 and HCG 34. Top DBR is not necessary.

When a voltage is applied, anchors do not allow current to flow over and cause HCG 34 to move. This creates charging between HCG 34 and the mesa 122. When voltage is increased, an attraction vis electro static actuation can be increased. As a non-limiting example, actuation is achieved with piezoelectric, thermal and the like. This provides tuning that depends of the wavelength and power. In one embodiment, tuning up to 100 nm is provided.

In one embodiment, electrostatic actuation is used by application of a voltage.

As a non-limiting example, less than 1% of the output beam is emitted from VCSEL laser 10.

The remaining portions of sacrificial layer 44 not removed provide anchoring points for HCG 34. As a non-limiting example, portions of the sacrificial layer 44 remain to provide anchoring in order for HCG 34 to move.

There can be two or more support elements coupled to a remaining portion of sacrificial layer 44. In one embodiment, four support elements are provided. Different directions of moving HCG 34 are provided. In various embodiments, HCG 34 can have a variety of different geometries, including but not limited to hexagonal, octagonal, a double frame design and the like.

In one embodiment, the output 38 of the VCSEL laser has a long wavelength, including but not limited to from 1 micron to 1.7 microns, and in one instance, 1.365 microns.

In one embodiment, the output 38 of the VCSEL laser is a long wavelength, at least partially created from indium phosphide structure or material in the VCSEL laser 10. As a non-limiting example, VCSEL laser 10 can include an indium phosphide substrate, or a substrate that at least partially includes indium phosphide.

In one embodiment, VCSEL laser 10 is coupled to or includes a top DBR 42 or high contrast grading (HCG) 34. As a non-limiting example, bottom DBR 12 is a semiconductor DBR or a combination of a semiconductor DBR with a dielectric coating. In this manner, VCSEL 10 can include a dielectric coating. As a non-limiting example, the dielectric coating improves a broadening of a tuning range of the VCSEL laser 10.

In one embodiment, VCSEL laser 10 operates in a single mode or a multi-mode operation. As a non-limiting example, dimensions of the aperture and HCG 34 are contributing factors to the single mode operation.

In one embodiment, VCSEL laser 10 can deploy multiple tunnel junctions to enhance the output 38 of VCSEL laser 10.

As a non-limiting example, buried tunnel junctions (BTJ) improve an energy efficiency of VCSEL laser 10.

As a non-limiting example, a wavelength of the VCSEL laser output 38 can be swept to provide improved resolution. In one embodiment, the VCSEL laser output 38 is swept by modulating an HCG grading 34 up and down, e.g, its up and down movement relative to a top of VCSEL laser 10, wherein when the HCG 34 moves closes to non-extended portion relative to a top of VCSEL laser 10, a wavelength of the output 38 changes and returns closer to an original output 38 of the VCSEL laser, without extension of the grading.

In one embodiment, a mem's structure is coupled to the HCG grading 34 or top DBR 42 to create a swept source. As a non-limiting example, modulating a VCSEL laser output 38, in combination with a sweeping of wavelengths of the VCSEL laser output 38 allows for higher resolution and reduces at least a portion of atmospheric interference of VCSEL laser operation.

In one embodiment, multiple tunnel junctions increase an optical power of the VCSEL laser 19. These junctions are provided in a body of the VCSEL laser 10.

In one embodiment, a semiconductor optical amplifier is included with a modulator to allow a swept source to be modulated. As a non-limiting example, the modulation of the VCSEL laser turning the VCSEL laser on and off. As a non-limiting example, the modulation is from about 1 to 50 G. In another embodiment, the modulation is greater than 50 G.

In one embodiment, a heterogeneous integration of a half-VCSEL to a Si sub mount is provided with MEMS HCG mirror and integrated detector. As a non-limiting example, the HCG is a meta-surface that can be designed and manufactured to work as a very thin and low loss highly reflective surface.

Manufacturing MEMS HCG on a separate Si substrate can simplify VCSEL manufacturing and allows for one or more of: optimization of laser parameters such as smaller mesa, simpler epitaxial growth; more effective current injection; better current and optical confinement; lower contact resistance, better thermal management. As a non-limiting example, MEMS manufacturing on Si allows for much more complex structures than MEMS on III-V compounds.

While III-V requires epitaxial growth and top-down processing, Si allows for: manufacturing using both bulk and surface MEMS concepts; MEMS HCG on Si can be optimized for larger movements with minimal mirror deformation; a Si sub mount can have an integrated detector under HCG MEMS mirror which can be used for power control of the VCSEL.

In one embodiment, eutectic bonding of half VCSEL to a Si sub mount with a tunable HCG and detector is provided. It is unique on manufacturing, compared to other approaches in the literature, and as a non-limiting example, the HCG is produced using surface MEMS technology on a Si wafer. As a non-limited example, integration is provided of a detector and an HCG mirror.

As a non-limiting example, the half-VCSEL has a partial top mirror or does not have a top mirror. In one embodiment, the minimal structure has a bottom DBR and active region only.

As a non-limiting example, with the present invention, lasing requires high internal reflectivity (usually R>99.5%) for both bottom and top mirrors. In one embodiment, an output of the VCSEL provides light transmitted through these high-reflective mirrors.

Manufacture half-VCSEL out of a III-V compound epitaxial structure (GaAs or InP).

As a non-limiting example, half-VCSEL processing consists on mesa etch (both for electrical current and optical field confinement), metal contacts (electrical current injection in the laser) and bonding pads.

As a non-limiting example, bonding pads and metal contacts for electrical current injection can share the same pad. In one embodiment, this shared pad is further connected to an external source through lines defined on Si submount.

As a non-limiting example, the Si sub mount is a substrate that serves as a base for bonding half-VCSEL. In one embodiment, the HCG MEMS is produced on the Si sub mount and acts as tunable mirror for a half-VCSEL. As a non-limiting example, the detector is integrated on Si, under Si HCG. This detector can be further used for power control of the integrated VCSEL.

As a non-limiting example, bonding process is done by flip chip half-VCSEL and bonding it on top of the HCG MEMS in order to make it a full-VCSEL (active region in between 2 mirrors).

In one embodiment, HCG actuation can be by one or more of: electrostatic, piezoelectric, thermal and the like. As a non-limiting example, the HCG can move up and down. Contacts can be provided on both sides (Si sub mount and half-VCSEL). This provides for push/pull actuation.

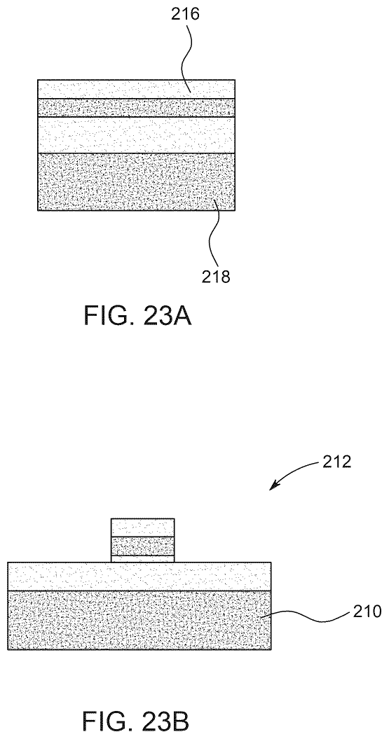

FIG. 23A illustrates one embodiment of a half-VCSEL 212 cross section of the present invention. FIG. 23B illustrates one embodiment of VCSEL 210 processing, of the present invention, with metal contacts (2) 214, mesa etch, 216 polyimide and bonding pads.

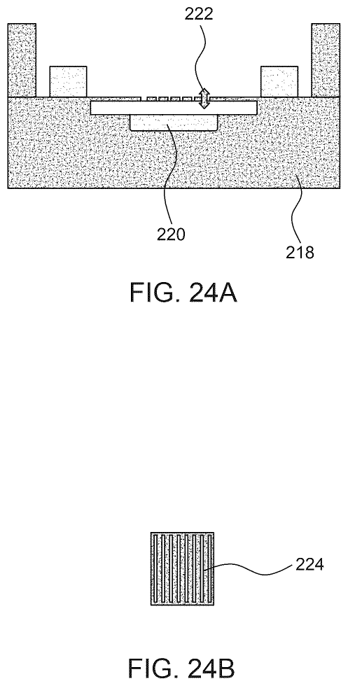

FIGS. 24A and 24B illustrate a cross-sectional view of a SI sub-mount 218. A SI SiGe detector 220 includes a—p-n junction with Ge diffusion into Si crystal. As a non-limiting example, the diameter can be about 30 um. Isolation of detector 220 can be achieved via amorphous Si deposition and SIOx deposition (sacrificial layer). A deposition of amorphous silicon can be used for HCG 222 manufacturing AuSn contacts can be provided for eutectic bonding. As a non-limiting example, Si bumps can be used for precise control of distance in between half VCSEL 212 and HCG 222.

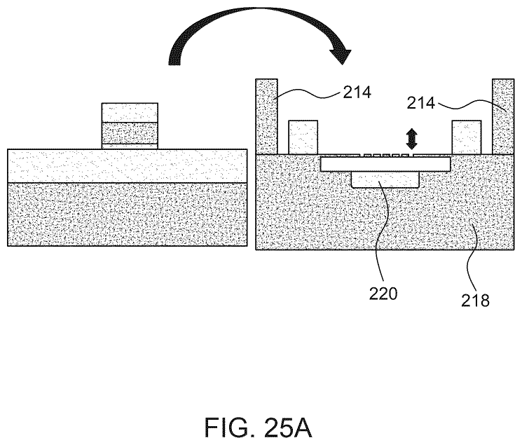

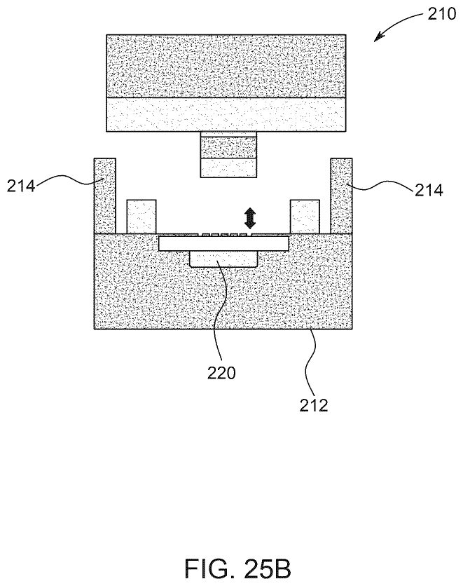

FIGS. 25A and 25B illustrates one embodiment of a flip chip 226 of the present invention.

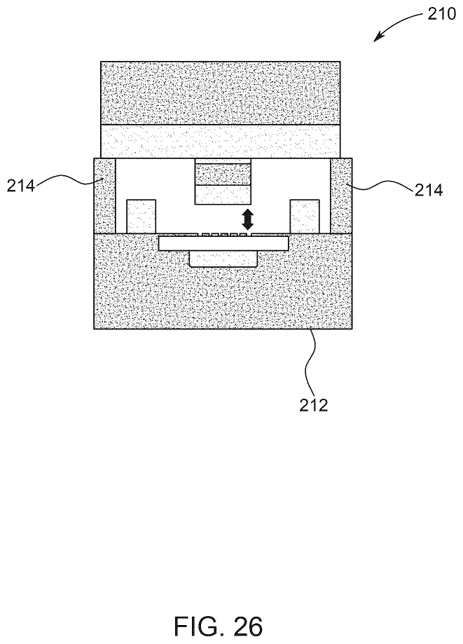

FIG. 26 illustrates one embodiment of a flip chip 226 bond of the present invention.

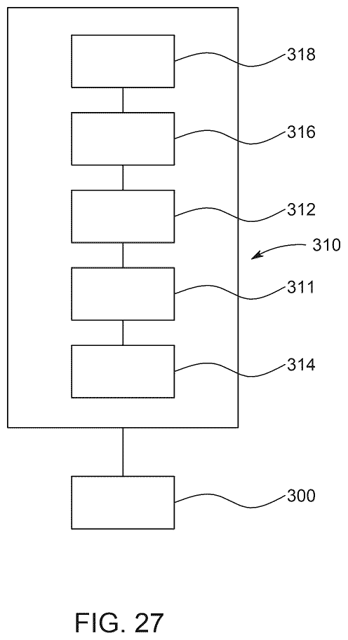

As illustrated in FIG. 27, VCSEL system 11 can use artificial intelligence (AI). Examples of AI used include but are not limited to including linear regression, logistic regression, decision tree, SVM algorithm, Naive Bayes algorithm, KNN algorithm, K-means, random forest algorithm, dimensionality reduction, and the like.

As a non-limiting example, a k-means algorithm is used.

In one embodiment, a user can seek from system a recommendation from the server and/or a recommendation engine 300. In one embodiment, the recommendation engine 300 make one or more of the following recommendations: regarding output readings of the VCSEL.

The recommendation engine 300 contains identifications and profiles of users who have posted recommendations/ratings, as well as profiles for users and usage feedback for videos and streamed media. A user seeking to use the recommendation engine 300 is presented (at some time) with a set of questions or the system otherwise obtains data inputs defining the characteristics of the user. In this case, the user characteristics generally define the context which is used to interpret or modify the basic goal of the user, and therefore the reference-user(s) for the user, though the user may also define or modify the context at the time of use. Various considerations are used in a cluster analysis, in which recommendations relevant to the contexts may be presented, with a ranking according to the distance function from the “cluster definition”. As discussed above, once the clustering is determined, advertisements may be selected as appropriate for the cluster, to provide a subsidy for operation of the system, and also to provide relevant information for the user about available products.

Clustering algorithms partition data into a certain number of clusters (groups, subsets, or categories). Important considerations include feature selection or extraction (choosing distinguishing or important features, and only such features); clustering algorithm design or selection (accuracy and precision with respect to the intended use of the classification result; feasibility and computational cost; etc.); and to the extent different from the clustering criterion, optimization algorithm design or selection.

Finding nearest neighbors can require computing the pairwise distance between all points. However, clusters and their cluster prototypes might be found more efficiently. Assuming that the clustering distance metric reasonably includes close points, and excludes far points, then the neighbor analysis may be limited to members of nearby clusters, thus reducing the complexity of the computation.

There are generally three types of clustering structures, known as partitional clustering, hierarchical clustering, and individual clusters. The most commonly discussed distinction among different types of clustering's is whether the set of clusters is nested or unnested, or in more traditional terminology, hierarchical or partitional. A partitional clustering is simply a division of the set of data objects into non-overlapping subsets (clusters) such that each data object is in exactly one subset. If the clusters have sub-clusters, then we obtain a hierarchical clustering, which is a set of nested clusters that are organized as a tree. Each node (cluster) in the tree (except for the leaf nodes) is the union of its children (sub-clusters), and the root of the tree is the cluster containing all the objects. Often, but not always, the leaves of the tree are singleton clusters of individual data objects. A hierarchical clustering can be viewed as a sequence of partitional clustering's and a partitional clustering can be obtained by taking any member of that sequence; i.e., by cutting the hierarchical tree at a particular level.

There are many situations in which a point could reasonably be placed in more than one cluster, and these situations are better addressed by non-exclusive clustering. In the most general sense, an overlapping or non-exclusive clustering is used to reflect the fact that an object can simultaneously belong to more than one group (class). A non-exclusive clustering is also often used when, for example, an object is “between” two or more clusters and could reasonably be assigned to any of these clusters. In a fuzzy clustering, every object belongs to every cluster with a membership weight. In other words, clusters are treated as fuzzy sets. Similarly, probabilistic clustering techniques compute the probability with which each point belongs to each cluster.

In many cases, a fuzzy or probabilistic clustering is converted to an exclusive clustering by assigning each object to the cluster in which its membership weight or probability is highest. Thus, the inter-cluster and intra-cluster distance function is symmetric. However, it is also possible to apply a different function to uniquely assign objects to a particular cluster.

A well-separated cluster is a set of objects in which each object is closer (or more similar) to every other object in the cluster than to any object not in the cluster. Sometimes a threshold is used to specify that all the objects in a cluster must be sufficiently close (or similar) to one another. The distance between any two points in different groups is larger than the distance between any two points within a group. Well-separated clusters do not need to be spherical, but can have any shape.

If the data is represented as a graph, where the nodes are objects and the links represent connections among objects, then a cluster can be defined as a connected component; i.e., a group of objects that are significantly connected to one another, but that have less connected to objects outside the group. This implies that each object in a contiguity-based cluster is closer to some other object in the cluster than to any point in a different cluster.

A density-based cluster is a dense region of objects that is surrounded by a region of low density. A density-based definition of a cluster is often employed when the clusters are irregular or intertwined, and when noise and outliers are present. DBSCAN is a density-based clustering algorithm that produces a partitional clustering, in which the number of clusters is automatically determined by the algorithm. Points in low-density regions are classified as noise and omitted; thus, DBSCAN does not produce a complete clustering.

A prototype-based cluster is a set of objects in which each object is closer (more similar) to the prototype that defines the cluster than to the prototype of any other cluster. For data with continuous attributes, the prototype of a cluster is often a centroid, i.e., the average (mean) of all the points in the cluster. When a centroid is not meaningful, such as when the data has categorical attributes, the prototype is often a medoid, i.e., the most representative point of a cluster. For many types of data, the prototype can be regarded as the most central point. These clusters tend to be globular. K-means is a prototype-based, partitional clustering technique that attempts to find a user-specified number of clusters (K), which are represented by their centroids. Prototype-based clustering techniques create a one-level partitioning of the data objects. There are a number of such techniques, but two of the most prominent are K-means and K-medoid. K-means defines a prototype in terms of a centroid, which is usually the mean of a group of points, and is typically applied to objects in a continuous n-dimensional space. K-medoid defines a prototype in terms of a medoid, which is the most representative point for a group of points, and can be applied to a wide range of data since it requires only a proximity measure for a pair of objects. While a centroid almost never corresponds to an actual data point, a medoid, by its definition, must be an actual data point.

In the K-means clustering technique K initial centroids are selected, the number of clusters desired. Each point in the data set is then assigned to the closest centroid, and each collection of points assigned to a centroid is a cluster. The centroid of each cluster is then updated based on the points assigned to the cluster. We iteratively assign points and update until convergence (no point changes clusters), or equivalently, until the centroids remain the same. For some combinations of proximity functions and types of centroids, K-means always converges to a solution; i.e., K-means reaches a state in which no points are shifting from one cluster to another, and hence, the centroids don't change. Because convergence tends to b asymptotic, the end condition may be set as a maximum change between iterations. Because of the possibility that the optimization results in a local minimum instead of a global minimum, errors may be maintained unless and until corrected. Therefore, a human assignment or reassignment of data points into classes, either as a constraint on the optimization, or as an initial condition, is possible.

To assign a point to the closest centroid, a proximity measure is required. Euclidean (L2) distance is often used for data points in Euclidean space, while cosine similarity may be more appropriate for documents. However, there may be several types of proximity measures that are appropriate for a given type of data. For example, Manhattan (L1) distance can be used for Euclidean data, while the Jaccard measure is often employed for documents. Usually, the similarity measures used for K-means are relatively simple since the algorithm repeatedly calculates the similarity of each point to each centroid, and thus complex distance functions incur computational complexity. The clustering may be computed as a statistical function, e.g., mean square error of the distance of each data point according to the distance function from the centroid. Note that the K-means may only find a local minimum, since the algorithm does not test each point for each possible centroid, and the starting presumptions may influence the outcome. The typical distance functions for documents include the Manhattan (L1) distance, Bregman divergence, Mahalanobis distance, squared Euclidean distance and cosine similarity.

An optimal clustering will be obtained as long as two initial centroids fall anywhere in a pair of clusters, since the centroids will redistribute themselves, one to each cluster. As the number of clusters increases, it is increasingly likely that at least one pair of clusters will have only one initial centroid, and because the pairs of clusters are further apart than clusters within a pair, the K-means algorithm will not redistribute the centroids between pairs of clusters, leading to a suboptimal local minimum. One effective approach is to take a sample of points and cluster them using a hierarchical clustering technique. K clusters are extracted from the hierarchical clustering, and the centroids of those clusters are used as the initial centroids. This approach often works well, but is practical only if the sample is relatively small, e.g., a few hundred to a few thousand (hierarchical clustering is expensive), and K is relatively small compared to the sample size. Other selection schemes are also available.

The space requirements for K-means are modest because only the data points and centroids are stored. Specifically, the storage required is O((m+K)n), where m is the number of points and n is the number of attributes. The time requirements for K-means are also modest-basically linear in the number of data points. In particular, the time required is O(I×K×m×n), where I is the number of iterations required for convergence. As mentioned, I is often small and can usually be safely bounded, as most changes typically occur in the first few iterations. Therefore, K-means is linear in m, the number of points, and is efficient as well as simple provided that K, the number of clusters, is significantly less than m.

Outliers can unduly influence the clusters, especially when a squared error criterion is used. However, in some clustering applications, the outliers should not be eliminated or discounted, as their appropriate inclusion may lead to important insights. In some cases, such as financial analysis, apparent outliers, e.g., unusually profitable investments, can be the most interesting points.

Hierarchical clustering techniques are a second important category of clustering methods. There are two basic approaches for generating a hierarchical clustering: Agglomerative and divisive. Agglomerative clustering merges close clusters in an initially high dimensionality space, while divisive splits large clusters. Agglomerative clustering relies upon a cluster distance, as opposed to an object distance. For example, the distance between centroids or medoids of the clusters, the closest points in two clusters, the further points in two clusters, or some average distance metric. Ward's method measures the proximity between two clusters in terms of the increase in the sum of the squares of the errors that results from merging the two clusters.

Agglomerative Hierarchical Clustering refers to clustering techniques that produce a hierarchical clustering by starting with each point as a singleton cluster and then repeatedly merging the two closest clusters until a single, all-encompassing cluster remains. Agglomerative hierarchical clustering cannot be viewed as globally optimizing an objective function. Instead, agglomerative hierarchical clustering techniques use various criteria to decide locally, at each step, which clusters should be merged (or split for divisive approaches). This approach yields clustering algorithms that avoid the difficulty of attempting to solve a hard combinatorial optimization problem. Furthermore, such approaches do not have problems with local minima or difficulties in choosing initial points. Of course, the time complexity of O(m2 log m) and the space complexity of O(m2) are prohibitive in many cases. Agglomerative hierarchical clustering algorithms tend to make good local decisions about combining two clusters since they can use information about the pair-wise similarity of all points. However, once a decision is made to merge two clusters, it cannot be undone at a later time. This approach prevents a local optimization criterion from becoming a global optimization criterion.

In supervised classification, the evaluation of the resulting classification model is an integral part of the process of developing a classification model. Being able to distinguish whether there is non-random structure in the data is an important aspect of cluster validation.

In one embodiment, a k-means algorithm is used as follows:

The K Means Clustering algorithm finds observations in a dataset that are like each other and places them in a set. The process starts by randomly assigning each data point to an initial group and calculating the centroid for each one. A centroid is the center of the group. Note that some forms of the procedure allow you to specify the initial sets.

Then the algorithm continues as follows:

It evaluates each observation, assigning it to the closest cluster. The definition of “closest” is that the Euclidean distance between a data point and a group's centroid is shorter than the distances to the other centroids.

When a cluster gains or loses a data point, the K means clustering algorithm recalculates its centroid.

The algorithm repeats until it can no longer assign data points to a closer set.

When the K means clustering algorithm finishes, all groups have the minimum within-cluster variance, which keeps them as small as possible. Sets with minimum variance and size have data points that are as similar as possible. There is variability amongst the characteristics in each cluster, but the algorithm minimizes it.

In short, the observations within a set should share characteristics. In some cases, the analysts might need to specify different numbers of groups to determine which value of K produces the most useful results.

In one embodiment, artificial intelligence engine 300, provides information relative to interaction of AI intelligence engine 300 As a non-limiting example, this can be monitoring information and includes associated predictive information to predict what will happen relative to the monitored body.

As a non-limiting example, the artificial intelligence engine 300 is used for systems with a deep learning networks with many layers. The layered network can process extensive amounts of data and determine the “weight” of each link in the network, e.g., for example, in an image recognition system, some layers of the neural network might detect individual features of a face, like eyes, nose, or mouth, while another layer would be able to tell whether those features appear in a way that indicates a face.

As will be appreciated by those skilled in the art, there are many different artificial intelligence engines 300 that can be trained to generate suitable output values for a range of input values; the neuro-fuzzy logic engine 300 is merely one embodiment.

The measurement data, the information feeds, and the output parameters may be used to train an artificial intelligence engine 300 to control the one or more devices in response to the measurement data and information feeds In one embodiment, artificial intelligence engines 300 can be trained to recognize temporal patterns.

In one embodiment, the measurement data, the information feeds, and the output parameters may be used to train an artificial intelligence engine 300 to control the one or more devices in response to the measurement data and information feeds.

A computing system 310 includes a logic subsystem 311 and a storage subsystem 312. Computing system 310 may further include an input subsystem 314, an output subsystem 316, a communication subsystem 318, and/or other components. In response to recommendation engine 300 and computing system 312, a physical change is made to system 10, that can include recommendations, the amount of energy delivered, the delivery of light, an interrogation is made, and the like.

Logic subsystem 311 may include one or more physical logic devices configured to execute instructions. For example, the logic subsystem may be configured to execute instructions that are part of one or more applications, services, programs, routines, libraries, objects, components, data structures, or other logical constructs. Such instructions may be implemented to perform a task, implement a data type, transform the state of one or more components, achieve a technical effect, or otherwise arrive at a desired result.

The logic subsystem 311 may include one or more processors (as an example of physical logic devices) configured to execute software instructions. Additionally, or alternatively, the logic subsystem may include one or more hardware and/or firmware logic machines (as an example of physical logic devices) configured to execute hardware or firmware instructions. Processors of the logic subsystem may be single-core or multi-core, and the instructions executed thereon may be configured for sequential, parallel, and/or distributed processing. Individual components of the logic subsystem may be distributed among two or more separate devices, which may be remotely located and/or configured for coordinated processing. Aspects of the logic subsystem may be virtualized and executed by remotely accessible, networked computing devices configured in a cloud-computing configuration.

Storage subsystem 312 includes one or more physical, non-transitory memory devices configured to hold instructions executable by the logic subsystem in non-transitory form, to implement the methods and processes described herein. When such methods and processes are implemented, the state of storage subsystem 312 may be transformed. e.g., to hold different data. Storage subsystem 312 may include removable and/or built-in devices. Storage subsystem 312 may include optical memory devices, semiconductor memory devices, and/or magnetic memory devices, among other suitable forms. Storage subsystem 312 may include volatile, nonvolatile, dynamic, static, read/write, read-only, random-access, sequential-access, location-addressable, file-addressable, and/or content-addressable devices. Aspects of logic subsystem 310 and storage subsystem 312 may be integrated together into one or more hardware-logic components. While storage subsystem 312 includes one or more physical devices, aspects of the instructions described herein alternatively may be propagated by a communication medium (e.g., an electromagnetic signal, an optical signal, etc.) that is not necessarily held by a physical device for a finite duration.

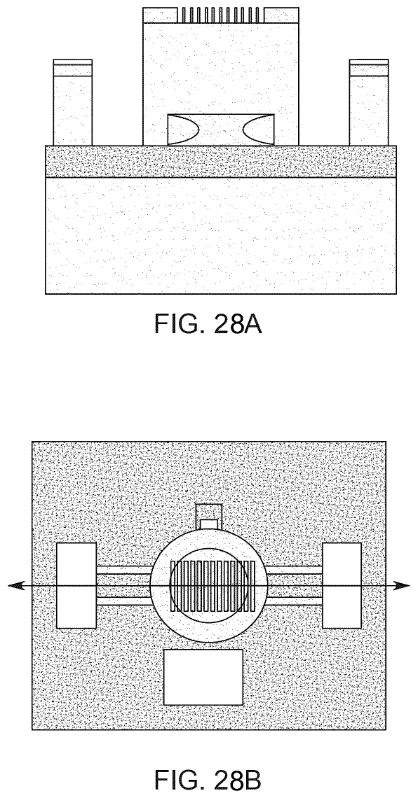

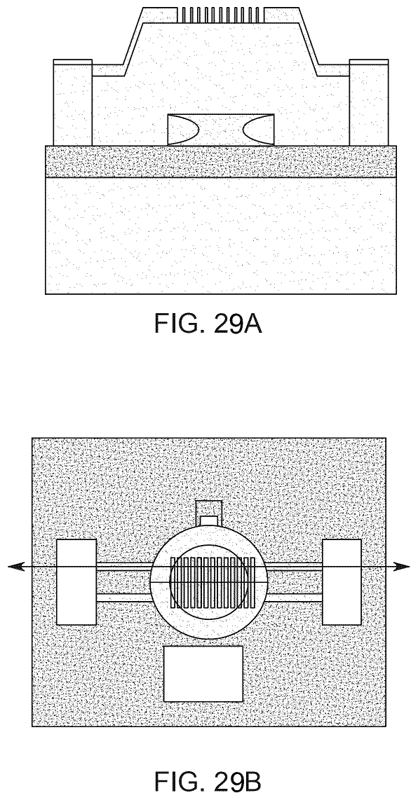

FIGS. 28A through 29B illustrate various embodiments of the present invention.

In one embodiment a regular VCSEL epitaxial structure includes a post etch between an active region and a sacrificial layer. A regrowth of sacrificial layer and HCG layer is done around the etched post. This provides a fully epitaxial grown tunable VCSEL with very small cavity volume, lateral electrical current and optical confinement, and thus, increased efficiency. The etched post and regrowth provide lateral current and optical confinement, small volume and increased efficiency for more demanding applications, such as very high-speed modulation and coherent communication.

Instead of etching a post for confinement in the optical path via mesa etch and regrowth, common approaches preserve the optical path and modify its boundaries for optical confinement. The existence of a sacrificial layer in the present invention favors this new approach as the final interface of regrown material in the optical path is etched away. This preserves optical quality. As a non-limiting example, the manufacturing requires integration and compatibility of several different processes, not required for the conventional semiconductor as-grown DBR.

Regrowth of the sacrificial layer around a small mesa step brings much smaller complication when compared to previous approaches of buried heterostructures with steep walls more than 2× taller (3-4 um) than our mesa (0.8 um).

In one embodiment, a full VCSEL is grown up to a thin SAC layer (100 nm of Al0.22Ga0.25In0.53As with 100 nm InP cap). Implantation is then proceeded for current confinement under the metal contact pads. The structure is then dry etch down a very tiny mesa how small for high-speed (radius of 5-10 um), up to InP layers, close to TJ interface (use optical monitoring). Atomic layer deposition (ALD) is performed only on side walls of the post. This protects the post for further release and provided increased reliability.

A full regrowth of a full sac layer+HCG on top is performed. A regrowth interface is etched away. The regrowth interface is out of optical/current paths. In one embodiment, the regrown layer is thick enough for relaxing defects before arriving at the HCG layer. In one embodiment, the mesa grows laterally during regrowth for a slightly bigger than mesa HCG. As a non-limiting example, a vertical step on the MEMS beams is not a problem. There is no need for implantation confinement.

In one embodiment, mesa confinement is provided, e.g, implantation is required only for contact isolation and current injection from the top of the mesa. In one embodiment, normal processing is completed for adding contact pads, which as a non-limiting example can be metal 3 and polyimide.

E-beam lithography has some topography on the MEMS beams. The beams can be defined by regular lithography (1-2 um wide).

The terms “module,” “program,” and “engine” may be used to describe an aspect of computing system 310 implemented to perform a particular function. In some cases, a module, program, or engine may be instantiated via logic subsystem 310 executing instructions held by storage subsystem 312. It will be understood that different modules, programs, and/or engines may be instantiated from the same application, service, code block, object, library, routine, API, function, etc. Likewise, the same module, program, and/or engine may be instantiated by different applications, services, code blocks, objects, routines, APIs, functions, etc. The terms “module,” “program,” and “engine” may encompass individual or groups of executable files, data files, libraries, drivers, scripts, database records, etc. A “service”, as used herein, may refer to an application program executable across multiple user sessions. A service may be available to one or more system components, programs, and/or other services. In some implementations, a service may run on one or more server-computing devices.

Each of the following references is expressly incorporated herein by reference in its entirety:
Abraham, Ittai, et al. “Low-distortion inference of latent similarities from a multiplex social network.” SIAM Journal on Computing 44.3 (2015): 617-668.
Aldenderfer, M. S., and R. K. Blashfield. Cluster Analysis. Sage Publications, Los Angeles, 1985.
Anderberg, M. R. (1973). Cluster Analysis for Applications. Academic Press, New York.
Anderson, E. (1957). A semi-graphical method for the analysis of complex problems. Proc. Nat. Acad. Sci. USA 43923-927.
Anderson, T. W. (1958). An Introduction to Multivariate Statistical Analysis. Wiley, New York.
Anderson, T. W., and Bahadur, R. R. (1962). classification into two multivariate normal distributions with different covariance matrices. Ann. Math. Statist. 33420-431.
Andrews, D. F. (1972). Plots of high-dimensional data. Biometrics 28 125-136.
Ankerst, M., M. M. Breunig, H. -P. Kriegel, and J. Sander. OPTICS: Ordering Points To Identify the Clustering Structure. In Proc. of 1999 ACM-SIGMOD Intl. Conf. on Management of Data, pages 49-60, Philadelphia, Pa., June 1999. ACM Press.
Arabie, P. (1977). clustering representations of group overlap. J. Math. Soc. 5 112-128.
Arabie, P. and Carroll, J. D. (1980). MAPCLUS: A mathematical programming approach to fitting to ADCLUS model. Psychometrika 45211-235.
Arabie, P., L. Hubert, and G. D. Soete. An overview of combinatorial data analysis. In P. Arabie, L. Hubert, and G. D. Soete, editors, Clustering and Classification, pages 188-217. World Scientific, Singapore, January 1996.
Art, D., Gnanadesikan, R., and Kettenring, J. R. (1982). Data-based metrics for cluster analysis. Utilitas Mathematica 31A 75-99.
Asimov, D. (1985). The grand tour. SLAM J. Sci. Statist. Corn-put. 6 128-143.
Auffarth, Benjamin, Yasumasa Muto, and Yasuharu Kunii. “An artificial system for visual perception in autonomous Robots.” Proceedings of the IEEE International Conference on Intelligent Engineering Systems. 2005.
Babu, B. Hari, N. Subash Chandra, and T. Venu Gopal. “Clustering Algorithms For High Dimensional Data—A Survey Of Issues And Existing Approaches.”
Baker, F. B. (1974). Stability of two hierarchical grouping techniques, Case I: Sensitivity to data errors. J. Amer. Statist. Assoc. 69440-445.
Ball, G., and D. Hall. A Clustering Technique for Summarizing Multivariate Data. Behavior Science, 12: 153-155, March 1967.
Banerjee, A., S. Merugu, I. S. Dhillon, and J. Ghosh. Clustering with Bregman Divergences. In Proc. of the 2004 SIAM Intl. Conf. on Data Mining, pages 234-245, Lake Buena Vista, Fla., April 2004.
Baraglia, R., Dazzi, P., Mordacchini, M., & Ricci, L. (2013). A peer-to-peer recommender system for self-emerging user communities based on gossip overlays. Journal of Computer and System Sciences, 79(2), 291-308.
Baragliaa, R., Dazzia, P., Mordacchinib, M., & Riccic, L. A Peer-to-Peer Recommender System for self-emerging user communities based on Gossip Overlays. (2012).
Beck, Carolyn, et al. “Dynamic Coverage and Clustering: A Maximum Entropy Approach.” Distributed Decision Making and Control. Springer London, 2012. 215-243.
Becker, P. (1968). Recognitions of Patterns. Polyteknisk, Copenhagen.
Bell, P. A. and Korey, J. L. (1975). QUICLSTR: A FORTRAN program for hierarchical cluster analysis with a large number of subjects. Behavioral Research Methods and Instrumentation 7575.
Berg, Mikko. “Human abilities to perceive, understand, and manage multi-dimensional information with visualizations.” (2012).
Birkin, P. Survey Of Clustering Data Mining Techniques. Technical report, Accrue Software, San Jose, Calif., 2002.
Bhat, Sajid Yousuf, and Muhammad Abolish. “A density-based approach for mining overlapping communities from social network interactions.” Proceedings of the 2nd International Conference on Web Intelligence, Mining and Semantics. ACM, 2012.
Binder, D. A. (1978). Comment on ‘Estimating mixtures of normal distributions and switching regressions’. j Amer. Statist. Assoc. 73746-747.
Blashfield, R. K., Aldenderfer, M. S. and Morey, L. C. (1982). cluster analysis literature on validation. In Classifying Social Data. (H. Hudson, ed.) 167-176. Jossey-Bass, San Francisco.
Bock, H. H. (1985). On significance tests in cluster analysis. J. Classification 277-108.
Boley, D. Principal Direction Divisive Partitioning. Data Mining and Knowledge Discovery, 2(4): 325-344, 1998.
Bosley, Daniel, and Vivian Borst. “A General Unsupervised Clustering Tool for Unstructured Data.” matrix 100: 2.
Boratto, Ludovico. “Group recommendation with automatic detection and classification of groups.” (2012).
Bradley, P. S. and U. M. Fayyad. Refining Initial Points for K-Means Clustering. In Proc. of the 15th Intl. Conf. on Machine Learning, pages 91-99, Madison, Wis., July 1998. Morgan Kaufmann Publishers Inc.
Breiman, L. Meisel, W. S., and Purcell, E. (1977). Variable kernel estimates of multivariate densities and their calibration. Technometrics 19 135-144.
Brineman, L., Friedman, J. H., Olshen, R. A., and Stone, C. J. (1984). Classification and Regression Trees. Wadsworth, Belmont, Calif.
Broadbent, S. R. and Hammersley, J. M. (1957). Percolation Processes, I: Crystals and Mazes. Proc. Cambridge Philos. Soc. 53629-641.
Bu, Yingyi, et al. “The HaLoop approach to large-scale iterative data analysis.” The VLDB Journal—The International Journal on Very Large Data Bases 21.2 (2012): 169-190.
Buja, A., Hurify, C. and Mcdonald, J. A. (1986). A data viewer for multivariate data. Computer Science and Statistics: Proceedings of the 18th Symposium on the Interface 171-174.
Cacoullos, T. (1966). Estimation of a multivariate density. Ann. Math. Statist. 18 179-189.
Cal, Rui, et al. “Scalable music recommendation by search.” Proceedings of the 15th international conference on Multimedia. ACM, 2007.
Carrizosa, Emilio, and Dolores Romero Morales. “Supervised classification and mathematical optimization.” Computers & Operations Research 40.1 (2013): 150-165.
Chang, Chin-Chun, and Hsin-Yi Chen. “Semi-supervised clustering with discriminative random fields.” Pattern Recognition 45.12 (2012): 4402-4413.
Chen, H., Gnanadesikan, R., and Kettenring, J. R. (1974). Statistical methods for grouping corporations. Sankhya B 36 1-28.
Chen, Yen Hung. “The k Partition-Distance Problem.” Journal of Computational Biology 19.4 (2012): 404-417.
Cheng, Hong, et al. “Clustering large attributed information networks: an efficient incremental computing approach.” Data Mining and Knowledge Discovery 25.3 (2012): 450-477.
Chernoff, H. (1972). The selection of effective attributes for deciding between hypotheses using linear discriminant functions. In Frontiers of Pattern Recognition. (S. Watanabe, ed.) 55-60. Academic Press, New York.
Chernoff, H. (1973a). Some measures for discriminating between normal multivariate distributions with unequal covariance matrices. In Multivariate Analysis Ill. (P. R. Krishnaiah, ed.) 337-344. Academic Press, New York.
Chernoff, H. (1973b). The use of faces to represent points in k-dimensional space graphically. J Amer. Statist. Assoc. 68 361-368.
Cherubini, Umberto, and Agnese Sironi. Bond Trading, Market Anomalies and Neural Networks: An Application with Kohonen Nets. No. ______012. Society for Computational Economics.
Christou, Ioannis T., George Gekas, and Anna Kyrikou. “A classifier ensemble approach to the TV-viewer profile adaptation problem.” International Journal of Machine Learning and Cybernetics 3.4 (2012): 313-326.
Clunies-Ross, C. W. and Riffenburgh, R. H. (1960). Geometry and linear discrimination. Biometrika 47185-189.
Cormack, R. M. (1971). A review of classification (with discussion). J Roy. Statist. Soc. A 134321-367.
Cover, T. M. (1968). Estimation by the nearest neighbor rule. IEEE Transactions Information Theory IT-14 50-55.
Cover, T. M. and Hart, P. E. (1967). Nearest neighbor pattern classification. IEEE Transactions, Information Theory IT-13 21-27.
Dallal, G. E. (1975) A user's guide to J. A. Hartigan's clustering algorithms. (unpublished manuscript) Yale University.
Day, N. E. (1969). Estimating the components of a mixture of normal distributions. Biometrika 56463-474.
Day, N. E., and Kerridge, D. F., (1967). A general maximum likelihood discriminant. Biometrics 23313-323. 94.
de Master, Trabajo Fin. “Novelty and Diversity Enhancement and Evaluation in Recommender Systems.” (2012).
Defays, D. (1977). An efficient algorithm for a complete link method. Computer Journal 20364-366.
Derrac, Joaquín, Isaac Triguero, Salvador García, and Francisco Herrera. “Integrading instance selection, instance weighting, and feature weighting for nearest neighbor classifiers by coevolutionary algorithms.”

It is to be understood that the present disclosure is not to be limited to the specific examples illustrated and that modifications and other examples are intended to be included within the scope of the appended claims. Moreover, although the foregoing description and the associated drawings describe examples of the present disclosure in the context of certain illustrative combinations of elements and/or functions, it should be appreciated that different combinations of elements and/or functions may be provided by alternative implementations without departing from the scope of the appended claims. Accordingly, parenthetical reference numerals in the appended claims are presented for illustrative purposes only and are not intended to limit the scope of the claimed subject matter to the specific examples provided in the present disclosure.

1. A VCSEL epitaxial structure, comprising:
an etched post between an active region and a sacrificial layer;
a regrowth of sacrificial layer and HCG layer around the etched post, providing a fully epitaxial grown tunable VCSEL with a cavity volume no greater than 50 cubic micrometers, lateral electrical current and optical confinement; and
wherein the etched post and regrowth provide lateral current and optical confinement, cavity volume small volume and an increased efficiency for applications of the VCSEL epitaxial structure.

2. The apparatus of claim 1, wherein a regrowth of the sacrificial layer and a HCG layer is around the etched post.

3. The apparatus of claim 2, regrowth of the sacrificial layer and a HCG layer is around the etched post provides a fully epitaxial grown tunable VCSEL with very small cavity volume, lateral electrical current and optical confinement.

4. The apparatus of claim 3, wherein the etched post and regrowth provides lateral current and optical confinement, small volume and increased efficiency for more demanding applications, such as very high-speed modulation and coherent communication.

5. The apparatus of claim 3, wherein the output of the VCSEL laser is a long wavelength, at least partially created from indium phosphide structure in the laser structure.

6. The apparatus of claim 4, wherein the VCSEL laser includes an indium phosphide substrate.

7. The apparatus of claim 1, wherein the VCSEL laser includes or is coupled to a top DBR or a high contrast grating (HCG).

8. The apparatus of claim 1, wherein a bottom DBR is a semiconductor DBR or a combination of a semiconductor DBR with a dielectric coating.

9. The apparatus of claim 1, wherein the VCSEL laser includes a dielectric coating.

10. The apparatus of claim 1, wherein the VCSEL laser operates in a single mode or a multi-mode operation.

11. The apparatus of claim 1, wherein the VCSEL laser operates in a single mode.

12. The apparatus of claim 10, wherein dimensions of the aperture and HCG are contributing factors to a single mode operation.

13. The apparatus of claim 13, wherein the VCSEL laser can deploy multiple tunnel junctions to enhance the output of the VCSEL laser.

14. The apparatus of claim 10.1, wherein the dielectric coating improves a broadening of a tuning range of the VCSEL laser.

15. The apparatus of claim 1, wherein buried tunnel junctions improve an energy efficiency of the VCSEL laser.

16. The apparatus of claim 1, wherein a wavelength of the VCSEL laser output can be swept to provide improved resolution.

17. The apparatus of claim 15, wherein the VCSEL laser output is swept by modulating a HCG grating up and down, wherein when the HCG moves closes to its non-extended original position, VCSEL the output wavelength(s) of the VCSEL laser changes and returns closer to an original output of the VCSEL laser when the HCG is not extended.

18. The apparatus of claim 8, further comprising:
a mem's structure coupled to the HCG grating or top DBR to create a swept source.

19. The apparatus of claim 1, wherein multiple tunnel junctions are provided that increase an optical power of the VCSEL laser.

20. The apparatus of claim 17, wherein a plurality of junctions are provided in a body of the VCSEL laser.
